(19) United States
(12) Patent Application Publication
(10) Pub. No.: US 2008/0312651 A1
(43) Pub. Date: Dec. 18, 2008

(54) APPARATUS AND METHODS FOR SELECTIVE HEATING OF TISSUE

(76) Inventors: Karl Pope, San Mateo, CA (US); Dean A. MacFarland, Magnolia, MA (US)

Correspondence Address: STALLMAN & POLLOCK LLP, 353 SACRAMENTO STREET , SUITE 2200, SAN FRANCISCO, CA, 94111, US

(21) Appl. No.: 11/764,094

(22) Filed: Jun. 15, 2007

(51) Int. Cl. A61B 18/14

(52) U.S. Cl. 606/42

(57) ABSTRACT

Methods and apparatus for selectively heating a target tissue via radiofrequency (RF) electrical energy. Apparatus of the invention includes an electrode unit having a plurality of concentric electrodes, wherein supply of electrical energy to each electrode may be independently controlled such that each of the plurality of electrodes has a different value of an electrical parameter for tissue treatment. Methods for selectively heating and treating tissue, for detecting thickness of tissue, and for determining a treatment value of an electrical parameter for each of a plurality of electrodes of an electrode unit are also disclosed.

### FIELD OF THE INVENTION

The present invention generally relates to apparatus and methods for treating tissue.

### BACKGROUND OF THE INVENTION

Adipose tissue (or “fat”) is an energy reserve in humans and other mammals. Fat is widely distributed subcutaneously (beneath the skin), although the thickness of the subcutaneous fat varies widely from person to person with differences associated with a number of factors including age, gender, diet, and lifestyle. In sedentary adults, subcutaneous fat may accumulate to excessive levels, especially in certain areas of the body, leading to obesity. Obesity is widespread in many societies and is considered a serious public health problem. Excessive body fat is known to predispose an individual to various diseases, including hypertension, diabetes, gallstones, sleep apnea, osteoarthritis, hernias, and cardiovascular disease. Obesity in humans may also lead to psychological as well as physical health problems. Accordingly, areas of excess fat accumulation may be targeted for the removal or destruction of adipose tissue, for example, via liposuction or lipolysis.

Liposuction, which involves the mechanical removal of adipose tissue from the body, has undesirable side-effects due to the invasive nature of such procedures. A number of procedures for disruption of adipose tissue, e.g., lipolysis induced by various energy sources including microwave, ultrasound, radiofrequency (RF), and laser energy, have been reported. Microwave, ultrasonic, and RF devices of the prior art have also been used in conjunction with liposuction to heat and soften adipose tissue so that the tissue can be more readily aspirated from adjacent tissue. However, conventional devices have experienced difficulty in controlling heat generation adjacent to the target site, which may result in undesirable collateral tissue damage. Furthermore, introduction of a suction device is invasive and can have significant or severe side-effects.

Prior art apparatus and methods for destroying adipose tissue are typically either invasive (e.g., call for insertion of apparatus through the patient's skin) and/or, in the case of prior art RF devices, have relied on capacitive coupling or inductive coupling to mitigate electrode edge effects, which lead to hot spots and collateral tissue damage. As an example, U.S. Pat. No. 5,143,063 to Fellner teaches supplying RF energy by capacitive coupling directly to the skin for areas close to the dermis via contact electrodes. The absorbed energy increases the temperature of the adipose tissue, and the adipose tissue is heated to an effective temperature (43.3-44.4° C.) for at least about 30-40 minutes. Fellner also teaches focusing energy, e.g., an incident ultrasonic wave, to a point within subcutaneous fat.

U.S. Pat. No. 6,413,255 to Stern discloses apparatus including various electrode configurations embedded in, coated with, or surrounded by dielectric or resistive materials, and a circular electrode divided into annular conductive rings in which current flow to inner and outer rings is controlled by a time sharing or duty cycle approach.

U.S. Pat. No. 4,527,550 to Ruggera et al. discloses an RF coil wound coaxially on a hollow support. The apparatus is constructed and operated to produce uniform deep-heating in tissue substantially axially located within the coil/support, and focuses heat along the coil's axis without excessively heating surface tissue.

U.S. Published Application No. 20060036300 discloses apparatus and a method for delivering RF energy below the skin surface to destroy fat cells. A region of skin is deformed so that the region protrudes out from surrounding skin. One or more RF electrodes are then applied to the skin protrusion to direct the RF current through the skin protrusion.

As can be seen, there is a need for apparatus and methods for selectively heating a target tissue in a non-invasive manner, such that the target tissue is effectively treated by the apparatus while adjacent non-target tissue remains essentially unchanged. There is a further need for apparatus and methods that rapidly heat target tissue without deforming target tissue or non-target tissue, while decreasing or eliminating the need for cooling fluids and cooling apparatus.

### SUMMARY OF THE INVENTION

In one aspect of the present invention, there is provided an electrosurgical system including a power supply and an electrode unit configured for coupling to the power supply. The electrode unit comprises a plurality of concentric electrodes, and the power supply is configured for supplying electrical energy to each of the plurality of concentric electrodes. The system is configured for independently controlling a first electrical parameter of the electrical energy supplied to each of the plurality of concentric electrodes, and the system is further configured for providing a different value of the first electrical parameter to each of the concentric electrodes.

In another aspect of the present invention, a system for treating a patient includes a power supply and an electrode unit including a plurality of concentric electrodes. The plurality of concentric electrodes includes a direct-coupled electrode and a plurality of indirect-coupled electrodes. The electrode unit is configured for direct electrical coupling of the direct-coupled electrode to the power supply, the electrode unit is further configured for electrical coupling of the direct-coupled electrode to each of the indirect-coupled electrodes, the power supply is configured for providing a supply of electrical energy to the electrode unit, and the system is configured for independently controlling the supply of electrical energy from the at least one direct-coupled electrode to each of the indirect-coupled electrodes.

In a further aspect of the present invention, a system for treating a patient includes an electrode unit having a plurality of concentric electrodes, and a power supply including a plurality of amplifiers. The electrode unit is configured for electrically coupling each of the plurality of concentric electrodes to a corresponding one of the plurality of amplifiers, and the power supply is configured for independently controlling supply of electrical energy to each of the plurality of concentric electrodes from the corresponding one of the plurality of amplifiers.

In still a further aspect of the present invention, there is provided a system including a power supply and an electrode unit operably coupled to the power supply. The electrode unit includes a plurality of concentric electrodes, and a plurality of passive electrical elements. Each of the electrodes is in electrical communication with the power supply via a corresponding one of the passive electrical elements, such that the system is configured for providing a different value of a first electrical parameter of electrical energy to each electrode.

In yet a further aspect of the present invention, an apparatus includes an electrode unit having a plurality of concentric annular electrodes, and a non-annular center electrode arranged concentrically with respect to each of the plurality of annular electrodes.

In still another aspect of the present invention, there is provided an apparatus including an electrode unit having a plurality of concentric electrodes. The plurality of concentric electrodes include a direct-coupled electrode and a plurality of indirect-coupled electrodes, the electrode unit is configured for direct electrical coupling of the power supply to the direct-coupled electrode, and the electrode unit is further configured for electrical coupling of the direct-coupled electrode to each of the indirect-coupled electrodes. The system is configured for independently controlling supply of electrical energy from the at least one direct-coupled electrode to each of the indirect-coupled electrodes.

In a further aspect of the present invention, a handpiece includes an electrode unit adapted for treating a patient's tissue. The electrode unit includes a plurality of concentric electrodes, and a treatment face configured for contacting the patient, wherein each of the plurality of concentric electrodes comprises a bare metal external surface, and the treatment face comprises the bare metal external surface.

In yet another aspect of the present invention, a method for treating a target tissue includes determining a treatment value of a first electrical parameter for each of a plurality of concentric electrodes of an electrode unit, wherein each concentric electrode has a different value of the first electrical parameter. The method further includes applying electrical energy to the target tissue via each of the concentric electrodes according to the predetermined treatment values.

In still another aspect of the present invention, a method for treating a patient includes disposing an electrode unit in relation to the patient's body, wherein the electrode unit includes a plurality of concentric electrodes. The electrode unit is electrically coupled to a power supply, the power supply includes a plurality of amplifiers, and each of the plurality of amplifiers is electrically coupled to a corresponding one of the plurality of concentric electrodes. The method further includes selectively heating a target tissue of the patient's body via the concentric electrodes, wherein supply of electrical energy to each of the plurality of concentric electrodes is independently controlled via the plurality of amplifiers.

In still a further aspect of the present invention, a method for performing a procedure includes disposing an electrode unit on or at a treatment area of a patient's skin, wherein the electrode unit includes a plurality of concentric electrodes. The method further includes applying electrical energy via the electrode unit to a target tissue located beneath the treatment area, wherein a first electrical parameter of the electrical energy supplied to each of the plurality of concentric electrodes is independently controlled, and wherein each of the concentric electrodes receives a different value of the first electrical parameter.

In still another aspect of the present invention, there is provided a method for determining a treatment value of an electrical parameter for each of a plurality of electrodes of an electrode unit. The method may include assigning a first magnitude, M₁, to a first electrode of the plurality of electrodes; assigning a second through n^{th} magnitude, M₂-Mₙ, for each of a second through n^{th} electrode of the electrodes, wherein each of the second through n^{th} magnitudes is derived from the first magnitude; and determining a first through n^{th} value, P₁-Pₙ, of the electrical parameter for a corresponding one of the first through n^{th} electrodes. Each of the first through n^{th} values, P₁-Pₙ, may be a function of a corresponding one of the first through n^{th} magnitudes M₁-Mₙ.

In still another aspect of the present invention, a method for adjusting a treatment parameter of an electrode unit includes monitoring at least a first electrical parameter of at least one electrode of the electrode unit; and adjusting at least a second electrical parameter of the at least one electrode in response to a change in the first electrical parameter.

In still another aspect of the present invention, a method for detecting tissue thickness includes maintaining at least a first electrical parameter at a constant level for at least one electrode of an electrode unit; monitoring at least a second electrical parameter for the at least one electrode; and, based on at least one change in the second electrical parameter, detecting a change in thickness of a target tissue.

These and other features, aspects, and advantages of the present invention may be further understood with reference to the drawings, description, and claims which follow.

### BRIEF DESCRIPTION OF THE DRAWINGS

FIG. 1 is a block diagram schematically representing an electrosurgical system for selectively heating a target tissue, according to an embodiment of the invention;

FIGS. 2A-B are block diagrams each schematically representing an electrosurgical handpiece, according to embodiments of the invention;

FIG. 3A schematically represents an electrode unit having a plurality of concentric electrodes, according to an embodiment of the invention;

FIG. 3B schematically represents an electrode unit having a plurality of concentric electrodes, according to another embodiment of the invention;

FIG. 4A is a block diagram schematically representing an electrosurgical system for independently controlling the supply of electrical energy to each of a plurality of concentric electrodes, according to an embodiment of the invention;

FIG. 4B is a block diagram schematically representing an electrosurgical system for independently controlling the supply of electrical energy to each of a plurality of concentric electrodes, according to another embodiment of the invention;

FIG. 5A is a schematic representation of an electrode unit, as seen in perspective view, according to an embodiment of the invention;

FIG. 5B is a schematic representation of an electrode unit, as seen in side view, according to the invention;

FIG. 5C is a schematic representation of an electrode unit, as seen in side view, according to another embodiment of the invention;

FIG. 5D is a plan view of an electrode unit having a plurality of concentric annular electrodes and a treatment face, according to one embodiment of the invention;

FIG. 5E is a plan view of an electrode unit having a non-annular center electrode, a plurality of concentric annular electrodes, and a treatment face, according to another embodiment of the invention;

FIGS. 5F-J each schematically represents a cross-section of an annular electrode of FIGS. 5D and 5E, according to various embodiments of the invention;

FIG. 5K schematically represents a center electrode for an electrode unit, according to an embodiment of the invention;

FIG. 6 schematically represents a portion of a disc-shaped electrode unit having a plurality of concentric annular electrodes and a non-annular center electrode, as seen in perspective view, according to an embodiment of the invention;

FIG. 7A schematically represents a transverse section of an electrode unit having a dielectric material disposed between concentric electrodes, according to one embodiment of the invention;

FIG. 7B schematically represents a transverse section of an electrode unit having a dielectric material disposed between concentric electrodes, according to another embodiment of the invention;

FIG. 8A is a block diagram schematically representing a system including an electrode unit having a center electrode coupled directly to a power supply, according to one embodiment of the invention;

FIG. 8B is a block diagram schematically representing a system including an electrode unit having an annular electrode coupled directly to a power supply, according to another embodiment of the invention;

FIG. 8C is a block diagram schematically representing a system including an electrode unit having electrodes coupled to a power supply via passive electrical elements, according to another embodiment of the invention;

FIG. 9A schematically represents a non-invasive procedure for selectively heating a target tissue via an electrosurgical system, according to an embodiment of the invention;

FIG. 9B schematically represents a non-invasive procedure for selectively heating a target tissue to provide a zone of maximum heating within the target tissue via an electrode unit disposed external to a patient's body, according to an embodiment of the invention;

FIG. 10 is a flow chart schematically representing a series of steps involved in a method for non-invasively treating tissue, according to another embodiment of the invention;

FIG. 11 is a flow chart schematically representing a series of steps involved in a method for non-invasively treating subcutaneous fat, according to another embodiment of the invention;

FIG. 12 is a flow chart schematically representing a series of steps involved in a method for determining a treatment value of an electrical parameter for each of a plurality of electrodes of an electrode unit, according to another embodiment of the invention;

FIG. 13 is a flow chart schematically representing a series of steps involved in a method for adjusting a value of a treatment parameter for an electrode unit during an electrosurgical procedure, according to another embodiment of the invention;

FIG. 14 is a flow chart schematically representing a series of steps involved in a method for detecting changes in thickness of a tissue, according to another embodiment of the invention;

FIG. 15 is a flow chart schematically representing a series of steps involved in a method for treating a target tissue, according to another embodiment of the invention; and

FIG. 16 schematically represents a temperature profile in a tissue section subjected to selective heating via an electrode unit, according to an embodiment of the invention.

### DETAILED DESCRIPTION OF THE INVENTION

The following detailed description is of the best currently contemplated modes of carrying out the invention. The description is not to be taken in a limiting sense, but is made merely for the purpose of illustrating the general principles of the invention, since the scope of the invention is best defined by the appended claims.

Broadly, the disclosed embodiments provide methods and apparatus for selectively heating a target tissue to a temperature sufficient to effectively treat the target tissue while an adjacent target tissue remains untreated and undamaged. These methods and apparatus may find applications, for example, in reducing the thickness of subcutaneous fat in a patient to achieve weight loss and decrease the numerous health risks associated with obesity. In another aspect of the disclosed methods, the thickness of subcutaneous fat may be decreased in one or more treatment areas of a patient's skin for aesthetic purposes. As an example, disclosed embodiments may be used to remove excess fat from a patient's thighs or abdomen to give a fitter, healthier, and younger appearance to the patient. In another example, embodiments may be used to remove excess fat from a patient's head or neck, e.g., around the chin, neck or eyelids, in a cosmetic procedure.

Unlike electrosurgical apparatus of the prior art, an embodiment as disclosed herein provides an electrode unit in the form of a plurality of concentric electrodes, wherein supply of electrical energy may be independently controlled to each of the plurality of concentric electrodes during an electrosurgical procedure, such that each electrode has a different value of at least one electrical parameter. The electrode unit may be substantially disc-shaped, and in some embodiments, may further include a non-annular center electrode. In contrast to the disclosed embodiments, prior art devices have used capacitive coupling or inductive coupling to prevent hot spots and prevent inadvertent burns to tissue. Apparatus of the type disclosed herein may avoid the use of cooling fluids and cooling sprays, and may be simpler and more economical to manufacture and operate, while providing less discomfort and collateral tissue damage to the patient, as compared with prior art devices.

While not being bound by theory, the Applicants have discovered that by using an electrode unit having multiple electrodes, e.g., in the form of a plurality of concentric electrically conducting metal rings, and by controlling the amount of voltage, current, or power that goes through each electrode or ring, it is possible to minimize the edge effect from any one ring, and hence from the electrode unit as a whole. By creating a gradient of voltage, current, or power, e.g., in which the energy levels are tapered radially from the innermost electrode to the outermost electrode, it is possible to achieve a much more even electric field within a tissue to be treated or contacted by apparatus of the type disclosed herein, as compared with a solid electrode, or even possibly a capacitively coupled electrode, of the prior art.

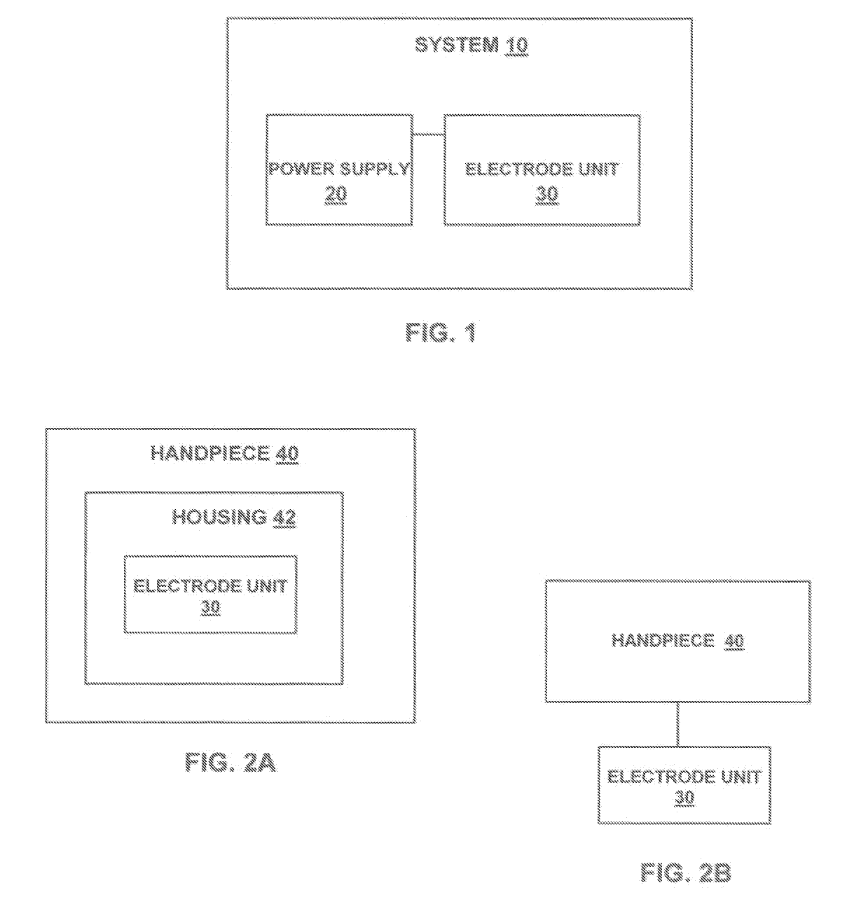

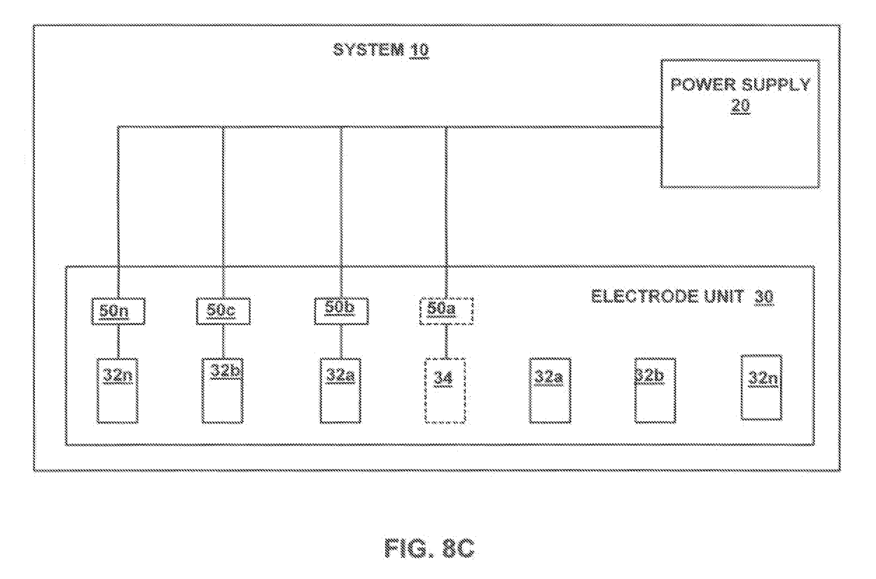

FIG. 1 is a block diagram schematically representing an electrosurgical system 10, according to an embodiment of the invention. Electrosurgical system 10 may be adapted or configured for treating, and selectively heating, a target tissue. System 10 may include a power supply 20 and an electrode unit 30. Power supply 20 may be an electrosurgical generator. Power supply 20 may be electrically coupled to electrode unit 30, e.g., via an electrical cord or cable 24 (see, for example, FIG. 9A). Target tissue is omitted FIG. 1 (see, for example, FIGS. 9A-B, and 15).

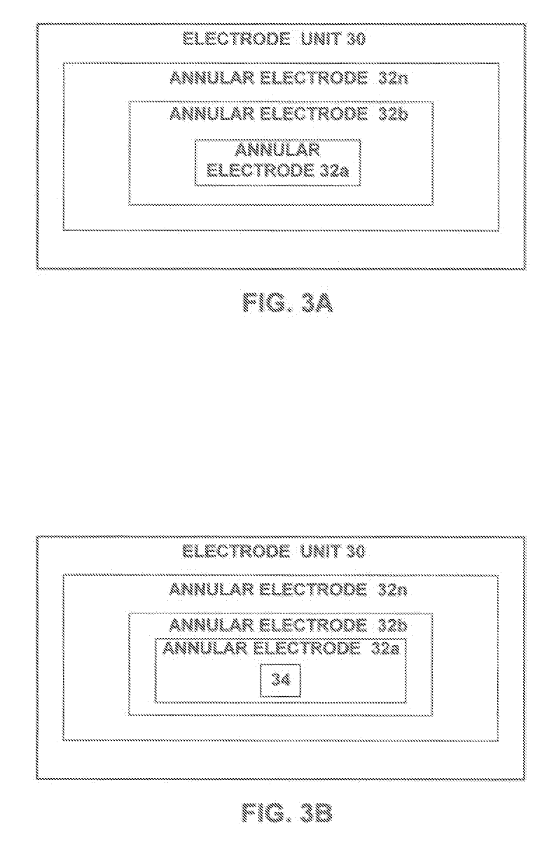

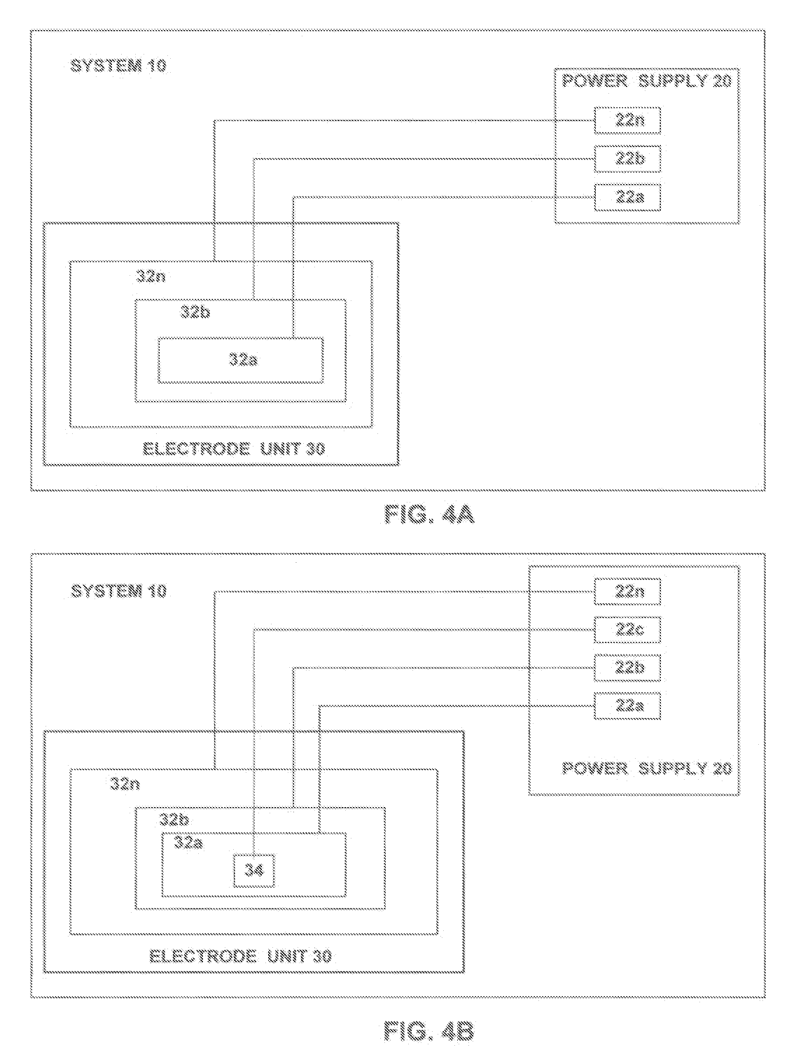

FIGS. 2A-B are block diagrams each schematically representing an electrosurgical handpiece 40, according to embodiments of the present invention. With reference to FIG. 2A, handpiece 40 may include a housing 42. Electrode unit 30 may be disposed at least partially within housing 42. Housing 42 may comprise a dielectric material, such as various polymers or plastic materials, as is well known in the art. In another configuration, see, e.g., FIG. 2B, electrode unit 30 may be otherwise affixed to, or integral with, handpiece 40. Electrode unit 30 may be configured for treating and selectively heating a target tissue of a patient (see, for example, FIGS. 3A-4B and 9B).

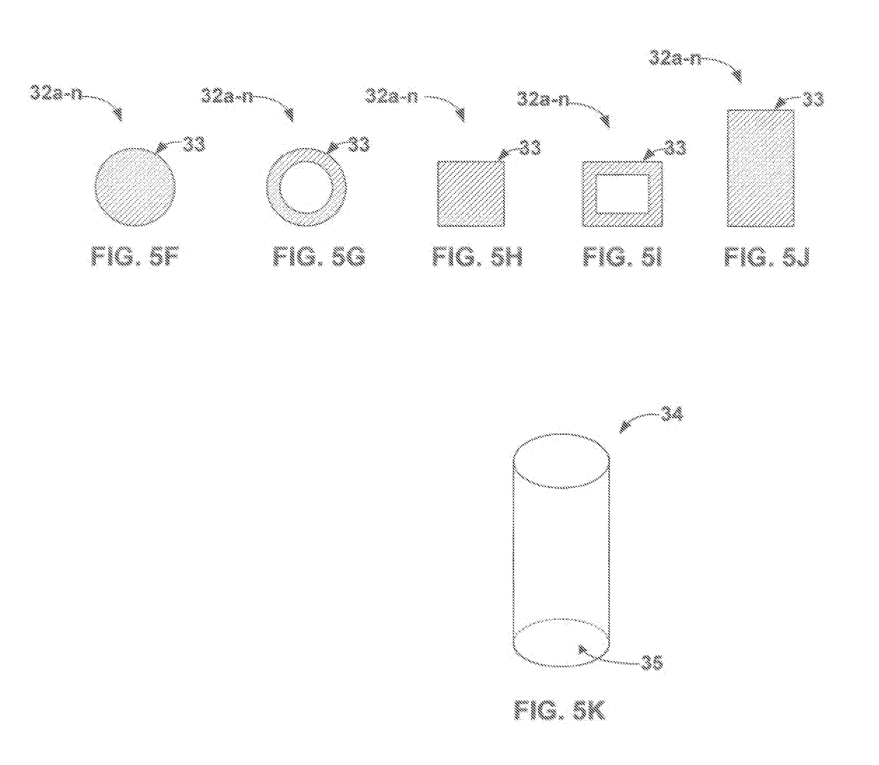

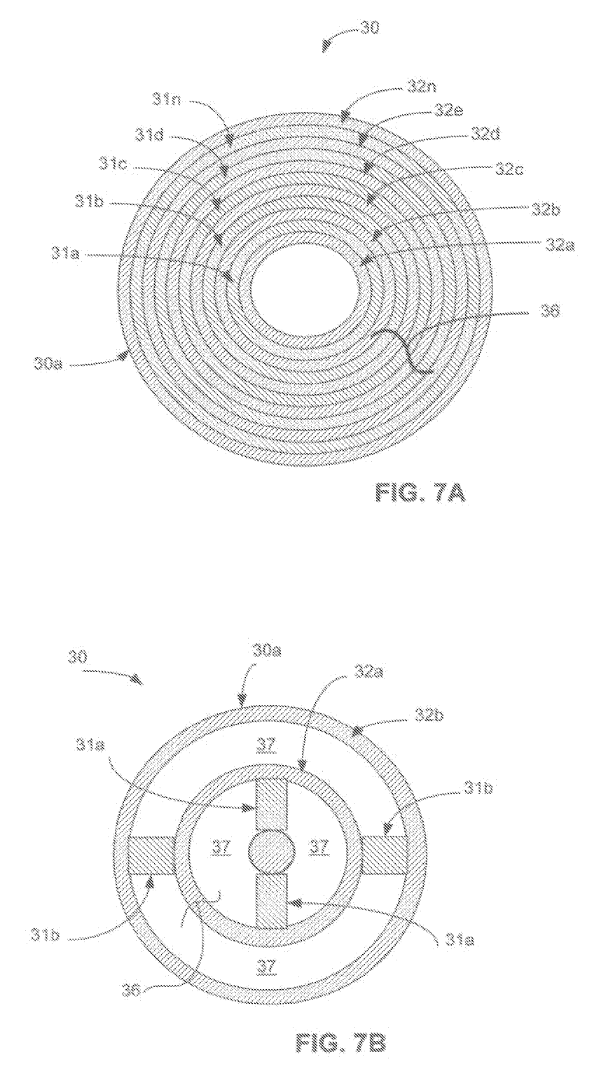

FIG. 3A is a block diagram schematically representing an electrode unit 30, according to an embodiment of the invention. Electrode unit 30 may include a plurality of concentric electrodes. In the embodiment of FIG. 3A, each of the concentric electrodes may be at least substantially annular (see, for example, FIGS. 5D and 7A). For example, as shown in FIG. 3A, electrode unit 30 may include a first annular electrode 32a, a second annular electrode 32b, and an nth annular electrode 32n. Each of the first through n^{th} annular electrodes 32a-n may have a different diameter (see, for example, FIGS. 5D-E). As non-limiting examples, electrode unit 30 may typically include at least about five (5) annular electrodes 32a-n, and up to about 25 or more annular electrodes 32a-n. In an embodiment, electrode unit 30 may include from about five (5) to eighteen (18) annular electrodes 32a-n, or from about six (6) to fifteen (15) annular electrodes 32a-n. In an embodiment, the n^{th} annular electrode 32n may be disposed radially outermost of the plurality of concentric electrodes 32a-n. A radially outward external surface of n^{th} annular electrode 32n may define an electrode perimeter 30a having a bare metal external surface (see, for example, FIGS. 5A, 5D-E).

FIG. 3B schematically represents an electrode unit 30, according to another embodiment of the invention. In the embodiment of FIG. 3B, electrode unit 30 includes a non-annular center electrode 34, in addition to a plurality of at least substantially annular electrodes, namely a first annular electrode 32a, a second annular electrode 32b, and an n^{th} annular electrode 32n. Center electrode 34 may be disposed at least substantially concentrically with respect to each of first through n^{th} annular electrodes 32a-n (see, for example, FIGS. 5E and 7B). In the embodiment of FIG. 3B, electrode unit 30 may include, as an example, electrode unit 30 may typically include at least about five (5) and up to about 25 or more annular electrodes 32a-n, or from about five (5) to eighteen (18) annular electrodes 32a-n, or from about six (6) to fifteen (15) annular electrodes 32a-n.

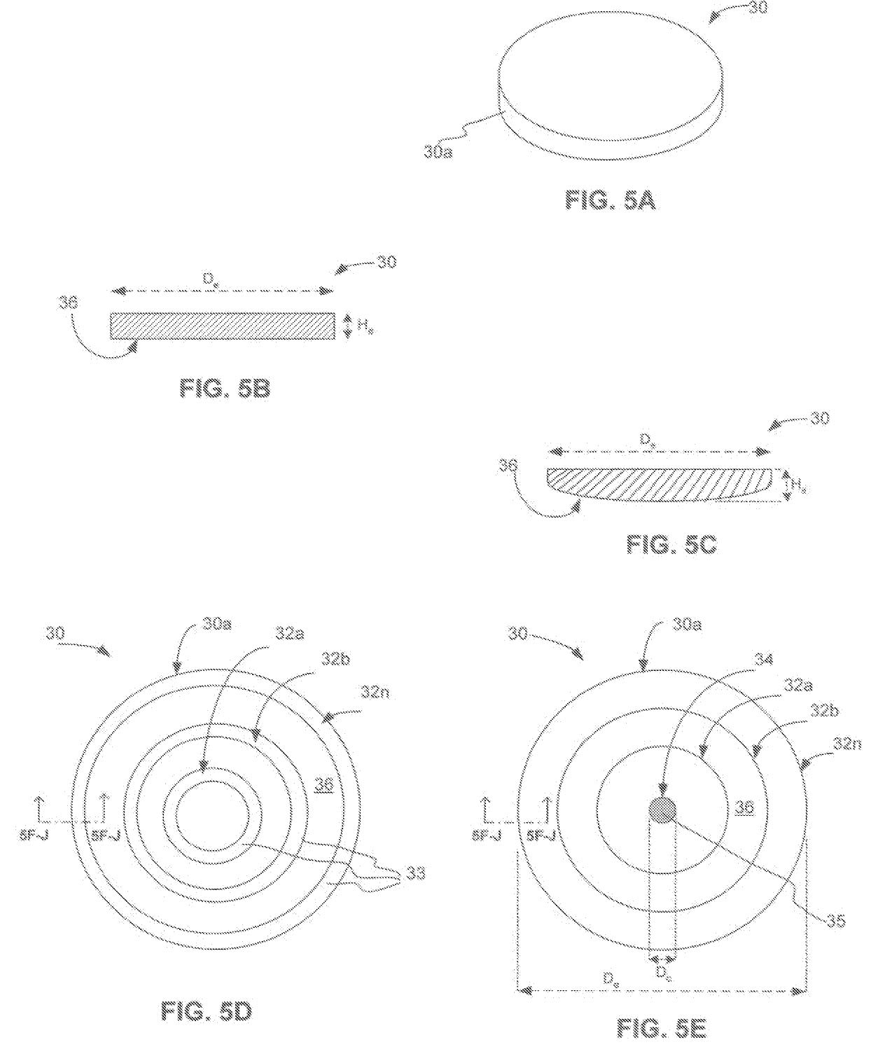

FIG. 4A is a block diagram schematically representing an electrosurgical system 10 for independently controlling the supply of electrical energy to each of a plurality of concentric electrodes, according to an embodiment of the invention. System 10 may include an electrode unit 30 and an electrosurgical generator or power supply 20. Electrode unit 30 may include a plurality of concentric annular electrodes, for example, first annular electrode 32a, second annular electrode 32b, and n^{th} annular electrode 32n, substantially as described with reference to FIG. 3A. Electrode unit 30 may be configured for electrical coupling to power supply 20. Power supply 20 may be configured for supplying electrical energy to electrode unit 30. System 10 may be configured for independently controlling the supply of electrical energy from power supply 20 to each of first annular electrode 32a, second annular electrode 32b, and n^{th} annular electrode 32n, such that each annular electrode 32a-n may have a different value of an electrical parameter for treating a patient's target tissue.

As shown in FIG. 4A, power supply 20 may include a plurality of amplifiers, namely, a first amplifier 22a, a second amplifier 22b, and an n^{th} amplifier 22n. First, second, and n^{th} annular electrodes 32a-n may be electrically coupled to first, second, and n^{th} amplifiers 22a-n, respectively, to allow for the independent control of at least one electrical parameter of each of first, second, and n^{th} annular electrodes 32a-n, wherein each annular electrode 32a-n may have a different value of the at least one electrical parameter during an electrosurgical procedure. As an example only, and not to limit the invention in any way, a value for current of each annular electrode 32a-n may be independently controlled during an electrosurgical procedure such that each annular electrode 32a-n has a different current value. Other configurations and mechanisms for independently controlling the supply of electrical energy to each of the plurality of concentric electrodes 32a-n are also within the scope of the invention.

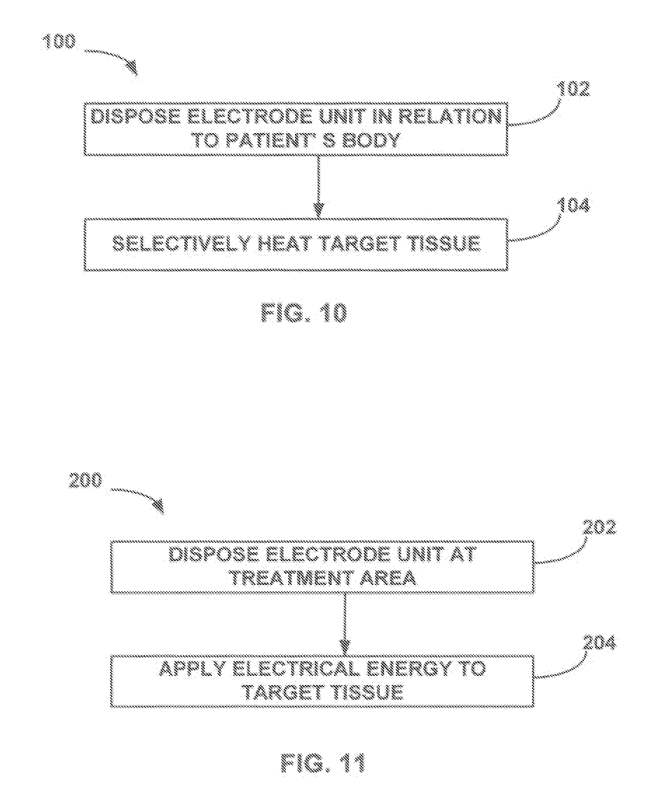

In an embodiment, different values for, e.g., current, or other electrical parameter(s), of each annular electrode 32a-n may be predetermined prior to treating a patient's tissue or prior to commencement of an electrosurgical procedure. Values for current, or other electrical parameter(s), of each annular electrode 32a-n may also be determined during a procedure for treating a patient's tissue. As an example only, a treatment value of an electrical parameter for each of first through n^{th} annular electrodes 32a-n may be determined based on an assigned magnitude for each of first annular electrode 32a, second annular electrode 32b, and n^{th} annular electrode 32n (see, for example, FIG. 12).

The use of different values for current, or other electrical parameter, of each electrode of a suitably configured electrode unit 30 may eliminate or greatly decrease an electrode edge effect. The use of different values for current, or other electrical parameter, of each electrode of a suitably configured electrode unit 30 may also allow for the controlled selective heating of a target tissue, e.g., subcutaneous fat, while electrode unit 30 is disposed on a non-target tissue, e.g., skin.

FIG. 4B is a block diagram schematically representing an electrosurgical system 10 for independently controlling the supply of electrical energy to each of a plurality of concentric electrodes, according to another embodiment of the invention. System 10 may include an electrode unit 30 and a power supply 20, substantially as described with reference to FIG. 4A. In the embodiment of FIG. 4B, however, electrode unit 30 may further include a center electrode 34 in addition to a plurality of annular concentric electrodes, i.e., first annular electrode 32a, second annular electrode 32b, and n^{th} annular electrode 32n.

With further reference to FIG. 4B, electrode unit 30 may be configured for electrical coupling to power supply 20, and power supply 20 may be configured for supplying electrical energy to electrode unit 30. In the embodiment of FIG. 4B, system 10 may be configured for independently controlling the supply of electrical energy from power supply 20 to center electrode 34 as well as to each of first annular electrode 32a, second annular electrode 32b, and n^{th} annular electrode 32n, wherein each of annular electrodes 32a-n and center electrode 34 has a different value of an electrical parameter for treating a patient's target tissue. Center electrode 34 may be a non-annular electrode, e.g., in the form of a pin, rod or metal post.

As shown in FIG. 4B, power supply 20 may include a plurality of amplifiers, namely, a first amplifier 22a, a second amplifier 22b, a third amplifier 22c, and an n^{th} amplifier 22n. In an embodiment, power supply 20 may include up to about twenty five (25) or more of amplifiers 22a-n, or from about five (5) to eighteen (18) amplifiers 22a-n. Each of the plurality of electrodes, including center electrode 34 and first through n^{th} annular electrodes 32a-n, may be independently coupled to a corresponding one of the plurality of amplifiers 22a-n. As a non-limiting example, center electrode 34 may be electrically coupled to third amplifier 22c, while first, second, and n^{th} annular electrodes 32a, 32b, 32n may be electrically coupled to first, second, and n^{th} amplifiers 22a, 22b, 22n, respectively. Independent electrical coupling of each of center electrode 34 and first through n^{th} annular electrodes to first through n^{th} amplifiers 22a-n allows for the independent control of at least one electrical parameter of each of center electrode 34 and first through n^{th} annular electrodes 32a-n, wherein each concentric electrode (i.e., center electrode 34 and annular electrodes 32a-n) has a different value of an electrical parameter for treating a patient. As an example only, and not to limit the invention in any way, values for current for center electrode 34 and for each of annular electrodes 32a-n may be independently controlled, via power supply 20, such that each of center electrode 34 and annular electrodes 32a-n has a different current value during an electrosurgical procedure. The different values for current, or other electrical parameter, of center electrode 34 and each annular electrode 32a-n may be predetermined, e.g., as an initial set of treatment values, prior to commencement of such a procedure. For example, the initial set of treatment values may be used during a first pass or treatment of a target tissue. During subsequent passes or treatments of the same or different target tissue(s), a second or subsequent set of treatment values may be determined for effectively treating the tissue.

The example given with respect to FIG. 4A for determining treatment values of an electrical parameter for each electrode may also be applicable to the embodiment of FIG. 4B. Similarly, a suitably configured electrode unit 30 having a center electrode 34 may eliminate or greatly decrease an electrode edge effect and allow for the selective heating of a target tissue, while eliminating the use of cooling mechanisms and cooling materials (e.g., refrigerant sprays).

FIG. 5A is a schematic representation of an electrode unit 30, as seen in perspective view, according to an embodiment of the invention. Electrode unit 30 may include an electrode perimeter 30a. Electrode perimeter 30a may comprise an electrically conductive metal. Electrode perimeter 30a may comprise a radially outward external surface of electrode unit 30, and the radially outward external surface of electrode unit 30 may be a bare metal external surface. As shown, electrode unit 30 may be at least substantially disc-shaped. Electrode perimeter 30a may be substantially circular.

FIG. 5B is a schematic representation of an electrode unit 30, as seen in side view, according to an embodiment of the invention. In the embodiment of FIG. 5B, electrode unit 30 may be flattened or disc-shaped, substantially as described with reference to FIG. 5A. Electrode unit 30 may have a height, Hₑ, and a diameter, Dₑ. Electrode unit 30 may include a treatment face 36. Treatment face 36 may be configured for contacting, or being disposed on, a portion of a patient's body (e.g., the external surface of the patient's skin) during a procedure. Treatment face 36 may be comprised of an external surface 33 of one or more concentric electrodes, such as annular electrodes 32a-n and/or center electrode 34 (see, for example, FIGS. 5D-E). Treatment face 36 may be at least substantially planar, as shown in FIG. 5B, or may have concave or convex curvature (see, for example, FIG. 5C).

FIG. 5C is a schematic representation of an electrode unit 30, as seen in side view, according to another embodiment of the invention. Electrode unit 30 may include a treatment face 36, which may be configured for contacting, or being disposed on, a portion of a patient's body during a procedure, and which may be comprised of an external surface 33 of one or more concentric electrodes, see, for example, FIG. 5D. As shown in FIG. 5C, treatment face 36 may have convex curvature. However, it is to be understood that other geometries for treatment face 36 are also within the scope of the present invention. As a non-limiting example, an electrode unit 30 having a curved treatment face 36 may have concentric electrodes occupying more than one plane.

FIG. 5D is a plan view of an electrode unit 30 showing a treatment face 36, according to an embodiment of the invention. As noted hereinabove, electrode unit 30 may include a plurality of concentric electrodes. More specifically, and as shown in FIG. 5D, electrode unit 30 may include first, second, and n^{th} annular electrodes 32a, 32b, 32n. In an embodiment, electrode unit 30 may include from about five (5) to about twenty five (25) annular electrodes 32a-n, typically from about five (5) to eighteen (18) annular electrodes 32a-n, and in some embodiments from about six (6) to fifteen (15) annular electrodes 32a-n. In an embodiment, all of the annular electrodes 32a-n may be in the same plane. In another embodiment, annular electrodes 32a-n may occupy more than one plane. Treatment face 36 may comprise an external surface 33 of each of the first through n^{th} annular electrodes 32a-n. Each of first, second, and n^{th} annular electrodes 32a, 32b, 32n may comprise an electrically conductive metal, such as stainless steel, aluminum, and various alloys, and the like; and external surface 33 may be a bare metal external surface. Electrode unit 30 may include an electrode perimeter 30a, which may comprise a radially outward external surface of electrode unit 30, and electrode perimeter 30a may comprise a bare metal external surface.

FIG. 5E is a plan view of an electrode unit 30 showing a treatment face 36, according to another embodiment of the invention. As shown in FIG. 5E, electrode unit 30 may comprise a plurality of concentric electrodes, including a non-annular center electrode 34 in addition to a plurality of concentric annular electrodes 32a-n. In an embodiment, center electrode 34 may be at least substantially cylindrical. As a non-limiting example, center electrode 34 may comprise a rod, a pin, or a post. Center electrode 34 may have a diameter, D_{c}, and electrode unit 30 may have a diameter, Dₑ.

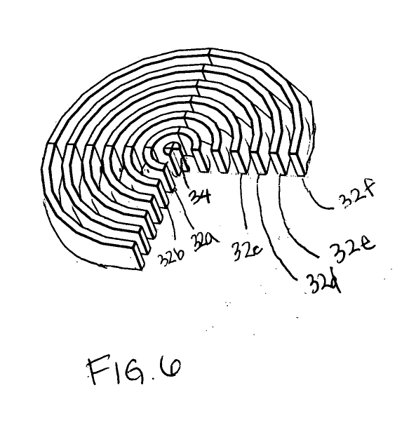

In the embodiment of FIG. 5E, treatment face 36 may comprise an external surface 35 of center electrode 34 in addition to an external surface 33 of each of the annular electrodes 32a-n. Center electrode 34 may comprise an electrically conductive metal, and external surface 35 may be a bare metal external surface. In an embodiment, all of the annular electrodes 32a-n as well as non-annular center electrode 34 may be in the same plane (see, for example, FIG. 6). In another embodiment, annular electrodes 32a-n and non-annular center electrode 34 may occupy more than one plane. External surfaces 33 of annular electrodes 32a-n are omitted from FIG. 5E for the sake of clarity. Electrode unit 30 may include an electrode perimeter 30a, essentially as described hereinabove. Electrode perimeter 30a may comprise a radially outward bare metal external surface of radially outermost annular electrode 32n.

FIGS. 5F-J each schematically represents a cross-section of annular electrodes 32a-n, as seen along the lines 5F-J-5F-J of FIGS. 5D and 5E, according to various embodiments of the invention. FIGS. 5F-J show a cross-section of an annular electrode 32a-n comprising, respectively, a substantially rounded solid wire, a substantially rounded hollow wire, a substantially square solid wire, a substantially square hollow wire, and a substantially rectangular solid wire or ribbon. Other cross-sectional configurations for annular electrodes 32a-n, including without limitation: solid wire or hollow wire having a generally triangular, polygonal, oval, or flattened cross-sectional shapes, and the like, are also within the scope of the invention. Annular electrode 32a-n may comprise an electrically conductive metal, e.g., in the form of a metal wire, a metal ribbon, or a length of metal tubing. Each of annular electrodes 32a-n shown in FIGS. 5F-J may include an external surface 33.

FIG. 5K schematically represents a non-annular center electrode 34, according to another aspect of the invention. As an example, center electrode 34 may be configured as an electrically conductive metal pin, rod, or post. Other geometries for center electrode 34 are also within the scope of the invention. Center electrode 34 may be disposed concentrically with respect to each of a plurality of annular electrodes 32a-n of electrode unit 30 (see, for example, FIGS. 6 and 7B). Center electrode 34 may have an external surface 35, which may be exposed on a treatment face 36 of electrode unit 30 (see, for example, FIG. 5E). Center electrode external surface 35 may be a bare metal external surface.

With further reference to FIGS. 5A-K, electrode unit 30 may be configured for treating and contacting tissue of a patient. Treatment face 36 may be configured for contacting a non-target tissue or a target tissue of a patient. For example, treatment face 36 may be configured for contacting a patient's skin or other tissue during treatment of the patient. Electrode unit 30 may be further configured for avoiding capacitive coupling and inductive coupling of electrode unit 30 to tissue, for example, such capacitive coupling and inductive coupling may be avoided when treatment face 36 is in contact with the patient's tissue during the selective heating of a target tissue by electrode unit 30.

FIG. 6 is a perspective view schematically representing an electrode unit 30, according to another embodiment of the invention. Electrode unit 30 may include a center electrode 34 and a first through a sixth annular electrodes 32a-f. Electrode unit 30 shown in FIG. 6 is sectioned for the sake of clarity of illustration. Electrode unit 30 may be adapted or configured for electrical coupling to a power supply 20 (see, e.g., FIGS. 1, 4A-B), such that electrical energy may be supplied independently to each of center electrode 34 and first through sixth annular electrodes 32a-f. The electrical energy may be supplied such that each of center electrode 34 and first through sixth annular electrodes 32a-f has a different value of an electrical parameter, such as current, voltage, or power, for treating tissue of a patient.

As shown in FIG. 6, each of first through sixth annular electrodes 32a-f may have a different diameter. The actual diameter of each annular electrode 32a-f, as well as the difference(s) in diameter between adjacent annular electrodes, may be varied, for example, according to the quality and quantity of a tissue to be treated, the nature of treatment to be performed, and the like. The characteristics of electrode unit 30 as described with reference to FIG. 6 may also be applicable to other embodiments of the present invention.

With further reference to FIG. 6, first through sixth annular electrodes 32a-f and center electrode 34 may each comprise an electrically conductive metal. Exemplary electrically conductive metals include stainless steel, aluminum, and various alloys, e.g., various alloys of aluminum, and the like. Center electrode 34 may be formed from a metal pin, post, or rod. Center electrode 34 may be disposed axially with respect to electrode unit 30. Center electrode 34 may be arranged concentrically with respect to each of first through sixth annular electrodes 32a-f.

In an embodiment, each of first through sixth annular electrodes 32a-f may comprise a metal ribbon, wherein the metal ribbon may be formed into a circular configuration. Such a metal ribbon may be oriented in various configurations with respect to a longitudinal axis of electrode unit 30. As a non-limiting example, a metal ribbon may be oriented longitudinally with respect to the longitudinal axis of electrode unit 30. Of course, alternative materials and techniques for forming each of first through sixth annular electrodes 32a-f, as may be apparent to the skilled artisan, are within the scope of the present invention.

Although, FIG. 6 shows electrode unit 30 having six annular electrodes 32a-f, other numbers and configurations for electrode unit 30 are also contemplated under the invention. As an example only, electrode unit 30 may include up to about 25 or more annular electrodes, while in some embodiments, electrode unit 30 may include from about five (5) to eighteen (18) annular electrodes 32a-n, or from about six (6) to fifteen (15) annular electrodes 32a-n. Furthermore, although FIG. 6 shows electrode unit 30 as having all six annular electrodes 32a-f and center electrode 34 in the same plane, embodiments having one or more concentric electrodes occupying different planes are also within the scope of the invention. For example, an electrode unit 30 having a convex treatment face 36 may have annular electrodes 32a-f and center electrode 34 occupying more than one plane.

FIG. 7A schematically represents a transverse section of an electrode unit 30 having a dielectric material disposed between concentric annular electrodes 32a-n, according to an embodiment of the invention. As shown in FIG. 7A, electrode unit 30 may include a plurality of concentric annular electrodes, 32a-n. Electrode unit 30 may include up to about twenty five (25) or more annular electrodes, 32a-n, in some embodiments from about five (5) to about eighteen (18) annular electrodes, 32a-n, or from about six (6) to about fifteen (15) annular electrodes, 32a-n. The dielectric material may be in the form of dielectric spacers 31 a-n. Dielectric spacers 31 a-n may be substantially annular, as shown; however, alternative configurations are also contemplated under the invention (see, for example, FIG. 7B infra). Electrode unit 30 may include an electrode perimeter 30a. Electrode perimeter 30a may comprise an electrically conductive metal surface of radially outermost annular electrode 32n. Electrode perimeter 30a may comprise a radially outward external surface of electrode unit 30, and the radially outward external surface of electrode unit 30 may be a bare metal external surface, i.e., free from dielectric material.

FIG. 7B schematically represents an axial section of an electrode unit 30 having a dielectric material disposed between concentric electrodes, according to another embodiment of the invention. As shown in FIG. 7B, dielectric spacers 31 a may be disposed between center electrode 34 and first annular electrode 32a. Dielectric spacers 31b may be disposed between first annular electrode 32a and second annular electrode 32b. Each of first and second annular electrodes 32a, 32b may comprise an electrically conductive metal. Electrode unit 30 may include an electrode perimeter 30a, which may comprise a radially outward external surface of electrode unit 30, and electrode perimeter 30a may comprise a bare metal external surface. Although only two annular electrodes 32a, 32b, are shown in FIG. 7B, other numbers of annular electrodes are also within the scope of the invention.

In the embodiment of FIG. 7B, dielectric spacers 31a and 31b may be in the form of rungs, or spokes. Such rungs or spokes may serve to support or brace annular electrodes 32a, 32b, and center electrode 34 with respect to each other. Dielectric spacers 31a-b may comprise one or more materials such as a ceramic, mica, glass, various plastics, or certain metal oxides, and the like. In some embodiments, there may be a void 37 between adjacent concentric electrodes 34. Such a void 37 may contain air or other gaseous dielectric materials. Alternatively, voids 37 may be evacuated and sealed to form a vacuum.

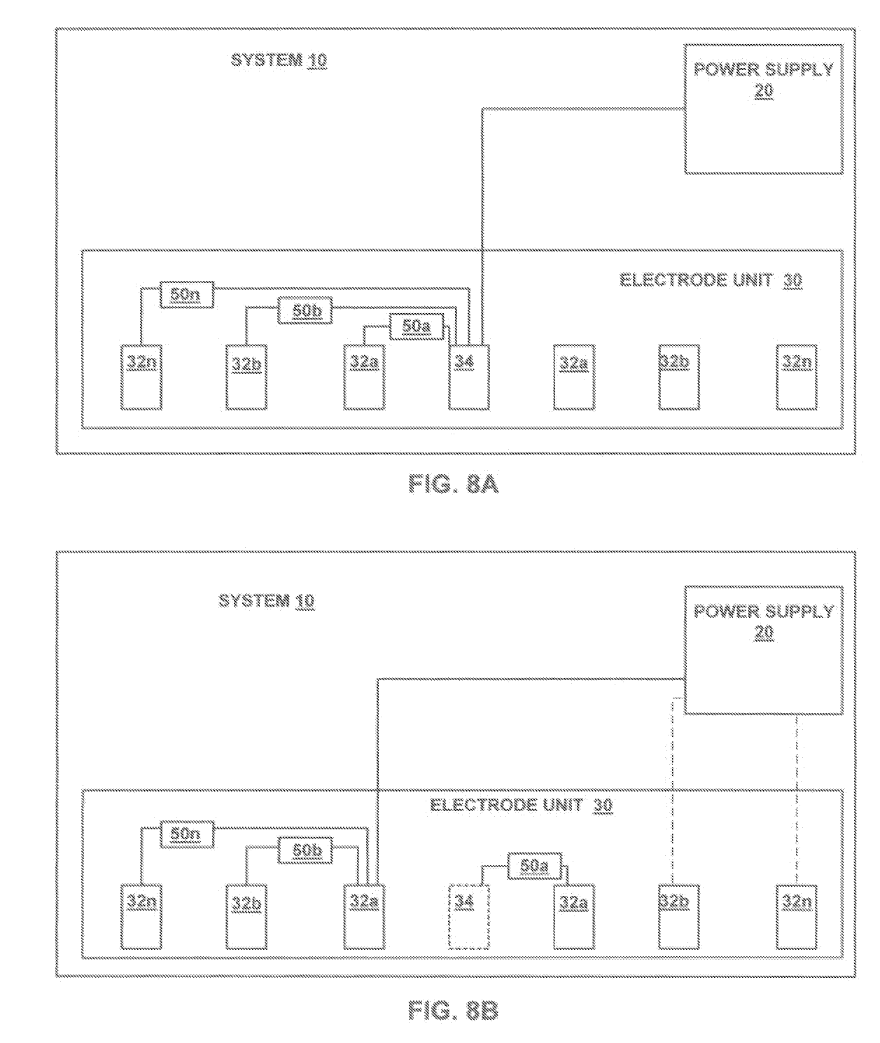

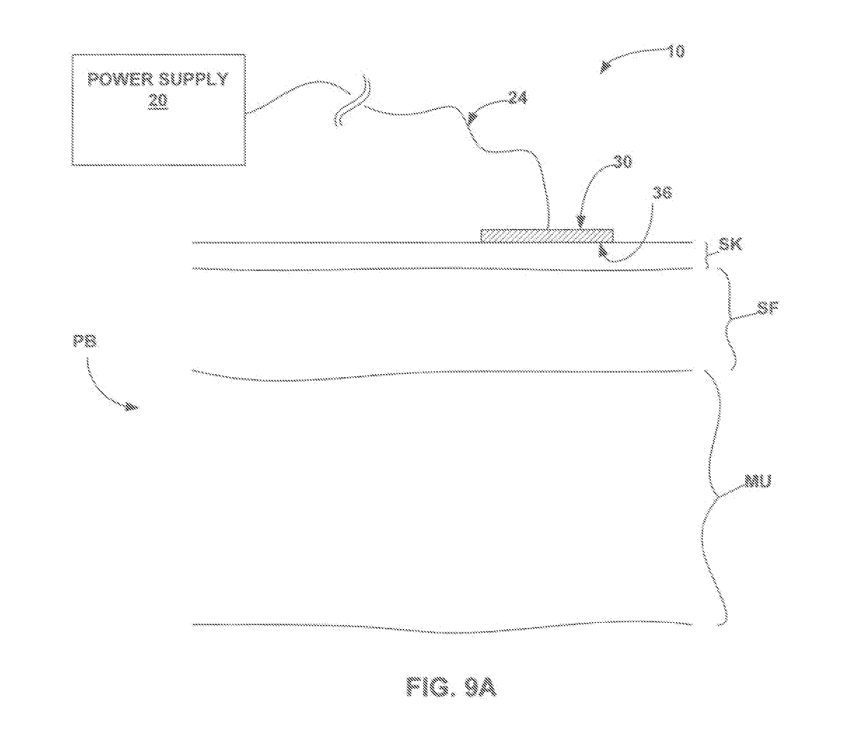

FIG. 8A is a block diagram schematically representing a system 10 including an electrode unit 30 having a center electrode 34 coupled directly to a power supply 20, according to another embodiment of the invention. System 10 may be configured and/or adapted for treating tissue of a patient, for example, as described herein with reference to one or more of FIGS. 10-15 (infra). For the embodiment of FIG. 8A, center electrode 34 may be referred to as a direct-coupled electrode. Electrode unit 30 may further include a plurality of annular electrodes, namely first, second, and n^{th} annular electrodes 32a, 32b, and 32n. For the embodiment of FIG. 8A, annular electrodes 32a-n may be referred to as indirect-coupled electrodes. Electrode unit 30 may still further include a plurality of passive electrical elements 50a-n. Each passive electrical element 50a-n may comprise, for example, a capacitor, a resistor, or an inductor. In an embodiment, each passive electrical element 50a-n may comprise, for example, at least one capacitor, at least one resistor, at least one inductor, or a combination thereof. As an example, one or more capacitors may be combined with one or more resistors and/or with one or more inductors to provide passive electrical elements 50a-n.

With further reference to FIG. 8A, electrode unit 30 may typically include from about five (5) to about 25 or more passive electrical elements 50a-n. In an embodiment, electrode unit 30 may include from about five (5) to eighteen (18) passive electrical elements 50a-n, or from about six (6) to fifteen (15) passive electrical elements 50a-n. Each of passive electrical elements 50a-n may have a different value of capacitance, inductance, or resistance. Each of center electrode 34 and first through n^{th} annular electrodes 32a-n may be arranged concentrically, substantially as described hereinabove, e.g., with reference to FIGS. 3B, 5E, 6, and 7B.

As shown in FIG. 8A, center electrode 34 may be electrically coupled to each of annular electrodes 32a-n via a corresponding one of passive electrical elements 50a-n. However, it is to be understood that alternative configurations are also within the scope of the invention. For example, in alternative embodiments or configurations, one of first, second, or n^{th} annular electrodes 32a, 32b, or 32n may be designated as a direct-coupled electrode for direct electrical coupling to power supply 20 (see, for example, FIG. 8B).

With still further reference to FIG. 8A, electrical energy supplied by power supply 20 may be distributed from direct-coupled center electrode 34 to indirect-coupled first through n^{th} annular electrodes 32a-n according to a respective value of passive electrical elements 50a-n. Each of passive electrical elements 50a-n may be selected to have a different value (capacitance, inductance, or resistance), such that each of first through n^{th} annular electrodes 32a-n and center electrode 34 has a different value of an electrical parameter, e.g., current, voltage, or power. As a non-limiting example, in an embodiment wherein passive electrical elements 50a-n comprise capacitors, capacitance values of elements 50a-n may decrease radially outward with respect to electrode unit 30; for example, a first capacitance of element 50a may be greater than a second capacitance of element 50b, which may in turn be greater than an n^{th} capacitance of element 50n. In an embodiment, the capacitance of elements 50a-n may typically be in the range of from about 0.1 to 10 picofarad (pF), usually from about 0.2 to 5 pF, and often from about 0.5 to 2 pF.

FIG. 8B is a block diagram schematically representing a system including an electrode unit 30 having a first annular electrode 32a coupled directly to power supply 20, according to another embodiment of the invention. Electrode unit 30 may further include a second through an n^{th} annular electrode 32b-n. In some embodiments, electrode unit 30 may still further include a center electrode 34, while in other embodiments center electrode 34 may be omitted. Electrode unit 30 may still further include a plurality of passive electrical elements, e.g., first, second, and n^{th} elements 50a, 50b, 50n, substantially as described with reference to FIG. 8A. In an embodiment, each passive electrical element 50a-n may comprise, for example, at least one capacitor, resistor, or inductor, or a combination thereof; and each passive electrical element 50a-n may have a different value of capacitance, inductance, or resistance. Each of annular electrodes 32a-n and center electrode 34 may be arranged concentrically, substantially as described hereinabove, e.g., with reference to FIGS. 3B, 5E, 6, 7B and 8A. In the embodiment of FIG. 8B, first annular electrode 32a is the only electrode that is electrically coupled directly to power supply 20; and, in the context of FIG. 8B, first annular electrode 32a may be referred to as a direct-coupled electrode. In alternative embodiments, second through n^{th} annular electrodes 32b-n may be designated for direct electrical coupling to power supply 20 (as represented by broken lines in FIG. 8B).

With further reference to FIG. 8B, direct-coupled first annular electrode 32a may be electrically coupled to each indirect-coupled annular electrode 32b-n via elements 50b-n. In embodiments wherein center electrode 34 is included in electrode unit 30, direct-coupled first annular electrode 32a may be similarly electrically coupled to center electrode 34 via element 50a. Electrical energy supplied by power supply 20 may be distributed from direct-coupled first annular electrode 32a to indirect-coupled second through n^{th} annular electrodes 32b-n, and in some embodiments to indirect-coupled center electrode 34, according to a respective capacitance, resistance, or inductance value of passive electrical elements 50a-n. Each of elements 50a-n may be selected to have a different capacitance, resistance, or inductance value, such that each of first through n^{th} annular electrodes 32a-n and center electrode 34 may have a different value of an electrical parameter, e.g., current, voltage, or power.

FIG. 8C is a block diagram schematically representing a system including an electrode unit 30 having electrodes coupled to a power supply 20 via passive electrical elements 50a-n, according to another embodiment of the invention. Electrode unit 30 may include a plurality of annular electrodes, e.g., first, and second through n^{th} annular electrodes 32a-n. Annular electrodes 32a-n may be concentrically arranged (see, for example, FIGS. 5D-E). Electrode unit 30 may further include a center electrode 34. Center electrode 34 may be concentrically arranged with respect to each of annular electrodes 32a-n. Center electrode 34 may be non-annular. As an example, center electrode 34 may be configured as a rod, a pin or a post (see, for example, FIGS. 5K and 6). Each of annular electrodes 32a-n and center electrode 34 may comprise an electrically conductive metal, and each of annular electrodes 32a-n and center electrode 34 may comprise a bare metal external surface.

Electrode unit 30 may still further include a plurality of passive electrical elements, e.g., first, and second through n^{th} passive electrical elements 50a, 50b, 50n, substantially as described with reference to FIG. 8A. Each passive electrical element 50a-n may comprise, for example, at least one capacitor, at least one resistor, at least one inductor, or a combination thereof, substantially as described with reference to FIG. 8A. Each of passive electrical elements 50a-n may have a different value of capacitance, inductance, or resistance, such that each of first through n^{th} annular electrodes 32a-n and center electrode 34 may have a different value of an electrical parameter, e.g., current, voltage, or power. In an embodiment, center electrode 34, and its associated passive electrical element 50a, may be optional.

With still further reference to FIGS. 8A-C, the number of annular electrodes 32a-n may vary, for example according to the composition and geometry of annular electrodes 32a-n, the presence or absence, composition, and geometry of center electrode 34, the values of passive electrical elements 50a-n, the size and curvature of a treatment face 36 (see, e.g., FIGS. 5D-E), the nature and thickness of a target tissue, the size of an area of skin to be treated, the values of electrical parameters to be provided to the annular electrodes 32a-n and center electrode 34 via power supply 20, etc. Typically, electrode unit 30 may include up to about 25 or more annular electrodes 32a-n, from about five (5) to about eighteen (18) annular electrodes 32a-n, or from about six (6) to about fifteen (15) annular electrodes 32a-n.

FIG. 9A schematically represents a non-invasive procedure for selectively heating a target tissue via an electrosurgical system 10, according to an embodiment of the invention. System 10 may include a power supply 20, which may be coupled to an electrode unit 30 via a suitable electrical cord or cable 24.

Power supply 20 and electrode unit 30 may each have elements, features, and characteristics as described herein with respect to various embodiments of the instant invention. As an example, electrode unit 30 may include a plurality of concentric electrodes (see, e.g., FIGS. 5D-E) and a treatment face 36 adapted for contacting a patient's body, PB. In an embodiment, electrode unit 30 may be a monopolar device. A return electrode (not shown), e.g., a dispersive pad, may be used during the procedure, as is well known in the art of electrosurgery. Electrode unit 30 may be disposed within, affixed to, or integral with, a handpiece 40 (see, for example, FIGS. 2A-B).

In an embodiment, treatment face 36 may be configured for contacting a patient's skin, SK. In an embodiment, treatment face 36 may be rigid. According to an aspect of the present invention, the patient's skin may represent a non-target tissue, and a target tissue may comprise a layer of subcutaneous fat, SF. That is to say, electrode unit 30 may contact the patient's skin for the purpose of treating adipose tissue underlying the skin. A layer of muscle underlying the subcutaneous fat may also represent non-target tissue. Electrical energy may be applied to the target tissue via concentric electrodes, e.g., annular electrodes 32a-n, comprising electrode unit 30. Applicant has found that by selecting a suitable configuration of concentric electrodes in combination with the astute selection, for each of the electrodes, of a suitable value of at least one electrical parameter, target tissue such as subcutaneous fat can be disrupted, in a non-invasive procedure, by selectively heating the target tissue without damaging adjacent non-target tissue, e.g., skin and muscle.

FIG. 9B schematically represents a procedure for non-invasively treating subcutaneous fat using an electrode unit 30, according to an embodiment of the invention. Electrode unit 30 may include a plurality of concentric electrodes, e.g., annular electrodes 32a-n, as described hereinabove with respect to various embodiments of the invention. Electrode unit 30 may further include a center electrode 34, which may be non-annular. Electrode unit 30 may still further include a treatment face 36. Treatment face 36 may comprise an external surface 33 of one or more annular electrodes 32a-n, and in some embodiments, an external surface 35 of center electrode 34 (see, for example FIGS. 5D, 5K). Treatment face 36 may be substantially planar (as shown in FIG. 9B), convex (see, e.g., FIG. 5C), or concave. In various embodiments, treatment face 36 may have other geometries or curvatures, and may be otherwise adapted for contacting an external surface of a patient's skin, SK. During a procedure for lipolysis of subcutaneous fat, treatment face 36 may be brought into direct contact with the external surface of the skin. In an embodiment, an electrically conductive fluid or gel (not shown) may be disposed between the skin and treatment face 36.

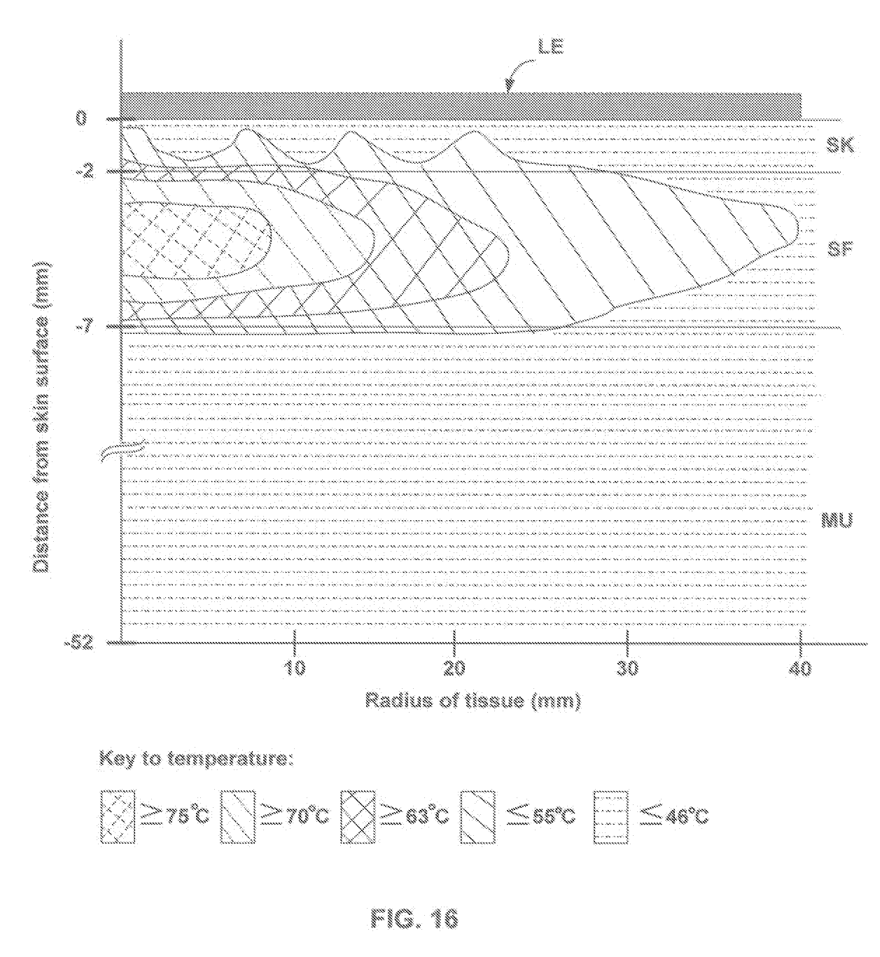

By the judicious selection of a value of at least one electrical parameter for each of the plurality of concentric electrodes, including center electrode 34 (when included), a zone of maximum heating, Tₘₐₓ, may be obtained within the layer of subcutaneous fat, SF, thereby inducing lipolysis of the subcutaneous fat while non-target tissue (skin and muscle, MU) remains intact and undamaged. The zone of maximum heating may be typically at least about 3 mm distant from treatment face 36, or at least about 3 mm beneath the external surface of the patient's skin. Usually the zone of maximum heating may be at least about 5 mm beneath the external surface of the patient's skin, and often about 7 mm or more beneath the external surface of the patient's skin. The zone of maximum heating may be controlled or adjusted depending on the thickness of the skin and/or the thickness of the subcutaneous fat. Thus, it can be seen that embodiments of the present invention may provide non-uniform heating in the Y dimension, i.e., in a direction substantially orthogonal to the layer of subcutaneous fat (see also, for example, FIG. 16). At the same time, embodiments of the invention may provide substantially uniform heating in the X and Z dimensions, within the layer of subcutaneous fat adjacent to electrode unit 30.

FIG. 10 is a flow chart schematically representing a series of steps involved in a method 100 for non-invasively treating tissue of a patient, according to another embodiment of the invention. Step 102 may involve disposing an electrode unit in relation to the patient's body. The electrode unit may have elements, features, and characteristics as described herein with respect to various embodiments of the invention. For example, electrode unit 30 may comprise a plurality of concentric electrodes, which may include a plurality of annular electrodes. In some embodiments, the plurality of concentric electrodes may further include a center electrode, wherein the center electrode may be non-annular. Typically, step 102 may involve disposing the electrode unit in at least close proximity to the patient's body and the electrode unit may be located external to the patient's body. Further, step 102 may involve disposing the electrode unit on a non-target tissue, wherein the target tissue may be disposed distal to the non-target tissue and distal to the electrode unit. As a non-limiting example, the non-target tissue may be the patient's skin, and in step 102 the electrode unit may be disposed on an external surface of the skin, while the target tissue may be subcutaneous fat disposed beneath the skin.

Step 104 may involve selectively heating, via the electrode unit, a target tissue of the patient's body while the electrode unit is disposed according to step 102. According to an aspect of the instant invention, such selective heating of target tissue may be obtained by defining or determining a different value of at least one electrical parameter for each of the plurality of concentric electrodes of the electrode unit (see, for example, FIG. 12). The supply of electrical energy to each of the plurality of concentric electrodes may be independently controlled according to the different values of the electrical parameter(s). Such an electrical parameter may be, for example, current, voltage, or power.

In an embodiment, the electrode unit may be moved in relation to regions of the target tissue to be treated during the procedure. The electrode unit may be affixed to or integral with a handpiece (see, e.g., FIGS. 2A-B). In one aspect of the invention, one or more electrical parameters may be monitored and adjusted during movement of the electrode unit with respect to regions of the target tissue.

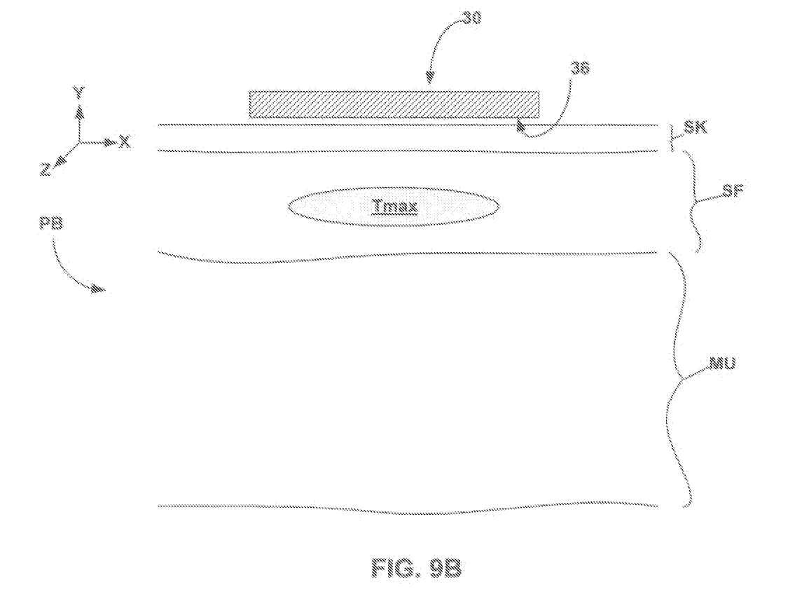

FIG. 11 is a flow chart schematically representing a series of steps involved in a method 200 for non-invasively treating subcutaneous fat, according to another embodiment of the invention. Step 202 may involve disposing an electrode unit at or on a treatment area of a patient's skin. The treatment area may comprise, for example, the thighs, abdomen, or neck of the patient. The electrode unit may be adapted for selectively applying electrical energy to targeted subcutaneous fat of the patient. The electrode unit may have certain elements and features as described hereinabove with respect to various embodiments of the present invention. For example, the electrode unit may comprise a plurality of concentric electrodes. The plurality of concentric electrodes may include a plurality of annular electrodes. The plurality of concentric electrodes may further include a non-annular center electrode.

During step 202, the patient's skin may be contacted with a treatment face of the electrode unit, wherein the treatment face may comprise an external surface of at least one of the plurality of concentric electrodes. Each of the plurality of concentric electrodes may comprise a bare metal external surface. In an embodiment, a fluid, gel, or other material may be applied to the patient's skin. Such material(s) may be applied to the patient's skin prior to or during steps 202 and 204. A fluid, gel, or other material applied to the patient's skin may comprise an electrically conductive material. In an embodiment, a material applied to the patient's skin may be an aqueous based-material, such as a dilute salt solution or hypotonic saline.

Step 204 may involve applying electrical energy to subcutaneous fat beneath the treatment area of the patient's skin. During step 204 the electrical energy may be applied through the patient's skin so as to target subcutaneous fat by selectively heating adipose tissue located beneath the patient's skin. As an example, in an embodiment the subcutaneous fat may be targeted at a depth of 3 mm to 7 mm or more beneath the surface of the patient's skin. A zone of maximum heating (see, for example, FIG. 9B) of the tissue may be located at a depth of 7 mm or more from the treatment face of the apparatus. The electrical energy may be applied to subcutaneous fat beneath a given treatment area of the patient's skin for a defined period of time, for example, by continuous or step-wise movement of the electrode unit from one treatment area to another treatment area of the patient's skin. Such a defined period of time for the application of electrical energy may typically be in the range of from about 1 second to 60 seconds, usually from about 3 seconds to 40 seconds, and often from about 5 seconds to 20 seconds.

With further reference to step 204, the application of electrical energy via the electrode unit may selectively heat the subcutaneous fat to a temperature sufficient to disrupt the fat tissue. A zone of maximum heating may be located within the subcutaneous fat beneath the electrode unit, such that non-target skin and muscle tissues are maintained at a relatively low temperature (see, for example, FIGS. 9B and 16). The electrode unit may be configured for minimizing heating of the patient's skin during step 204. As an example, in an embodiment at least a portion of the subcutaneous fat may be heated to a temperature of at least 50° C. as a result of step 204, whereas at the same time the patient's skin and muscle may be heated to a temperature of not more than about 47° C. In some embodiments, the subcutaneous fat may be heated to a temperature of at least 53° C., while the patient's skin and muscle may be heated to a temperature of not more than about 46° C. In other embodiments, the subcutaneous fat may be heated to a temperature of at least 55° C., while the patient's skin and muscle may be heated to a temperature of not more than about 44° C.

As a result of heating the subcutaneous fat as described with reference to step 204, lipolysis may be induced in at least a portion of adipocytes of the fat tissue.

In an embodiment, during or prior to step 204, a treatment value of an electrical parameter may be determined for each of the plurality of concentric electrodes, such that each of the plurality of concentric electrodes may have a different treatment value of the electrical parameter.

FIG. 12 is a flow chart schematically representing a series of steps involved in a method 300 for determining a treatment value of an electrical parameter for each of a plurality of concentric electrodes of an electrode unit, according to another embodiment of the invention. The electrode unit may have elements and characteristics as described herein for various embodiments of electrode unit 30, for example, as described with reference to FIGS. 1-9B (supra).

Step 302 may involve assigning a first magnitude, M₁, to a first electrode of the electrode unit. In an embodiment, the first electrode may be the innermost annular electrode (i.e., the annular electrode with the least diameter). In other embodiments, the first electrode may be an annular electrode disposed radially outward from the innermost annular electrode (i.e., an annular electrode with a diameter greater than that of the innermost annular electrode). In still other embodiments, the first electrode may be a center electrode, which may be non-annular, disposed axially or centrally within one or more annular electrodes. In an embodiment, the first magnitude may be assigned arbitrarily. As a non-limiting example, the first electrode may be arbitrarily assigned a magnitude of 1.0.

Step 304 may involve assigning a second through n^{th} magnitude, M₂-Mₙ, to a corresponding one of a second through n^{th} electrodes. Each of the second through n^{th} electrodes may be any electrode other than the first electrode. Each of the second through n^{th} magnitudes may be based on, or derived from, the first magnitude; and, each of the second through n^{th} magnitudes may be derived with respect to each other. For example, each of the second through n^{th} magnitudes may be a different fraction, or multiple, of the first magnitude. Each of the second through n^{th} magnitudes may be a function of the first magnitude. In an embodiment, for example, wherein the second electrode is radially outward from the first electrode and the n^{th} electrode is radially outward from the second electrode, M₁, M₂, and Mₙ may have the following relationship: M₁<M₂<Mₙ.

Step 306 may involve determining a first through n^{th} value, P₁-Pₙ, of an electrical parameter for a corresponding one of the first through n^{th} electrodes. For example, step 306 may include determining a second value, P₂, of the electrical parameter for a second electrode. The electrical parameter may be voltage, current, or power. Each of the first through n^{th} values may be a function of a corresponding magnitude. For example, the first through n^{th} values, respectively, may be a function of the first through n^{th} magnitudes assigned in steps 302 and 304. In an embodiment, each of the first through n^{th} values may be mathematically derived from the first through n^{th} magnitudes as a function of a scaling factor, S, and the area, A, of a circle defined by a particular annular electrode for which the value is to be determined. In an example wherein the electrical parameter may be current, the values of current, I₁-Iₙ for the first through n^{th} electrodes may be related to the magnitudes M₁-Mₙ by the relationship:

Iₓ=(Mₓ/Aₓ)*S,

where x denotes a particular one of the first through n^{th} electrodes (or x=1−n), Iₓ is current for the particular one of the first through n^{th} electrodes, Mₓ is magnitude for the particular one of the first through n^{th} electrodes, the particular one of the first through n^{th} electrodes is an annular electrode, Aₓ is the area of a circle defined by the particular one of the first through n^{th} electrodes, and S is the scaling factor. The scaling factor is the same (constant) for each electrode of a given electrode unit for a given treatment. However, the scaling factor may vary from treatment to treatment, for example, according to certain variables, including variables related to a patient to be treated by the electrode unit, such as a thickness or depth of a target tissue, and the like.

With further reference to method 300 (FIG. 12), in an embodiment the electrical parameter may be current, and the first through n^{th} values, P₁-Pₙ may be related as follows: P₁<P₂<Pₙ. In other embodiments, the electrical parameter may be voltage, and the first through n^{th} values, P₁-Pₙ may be related as follows: P₁>P₂>Pₙ.

In some embodiments, the electrode unit may include a non-annular center electrode (see, e.g., FIG. 5E). Step 308 may involve assigning a center magnitude, M_{c}, to the center electrode. The center magnitude may be assigned as a function of the first magnitude. In some embodiments, the center magnitude may be less than the first magnitude. As an example, M_{c} may be in the range of from about 0.01*M₁ to 0.95*M₁. In an embodiment, M_{c} may be in the range of from about 0.05*M₁ to 0.2*M₁. Step 310 may involve determining a center value, P_{c}, for the electrical parameter of the center electrode, wherein the center value may be a function of the center magnitude.

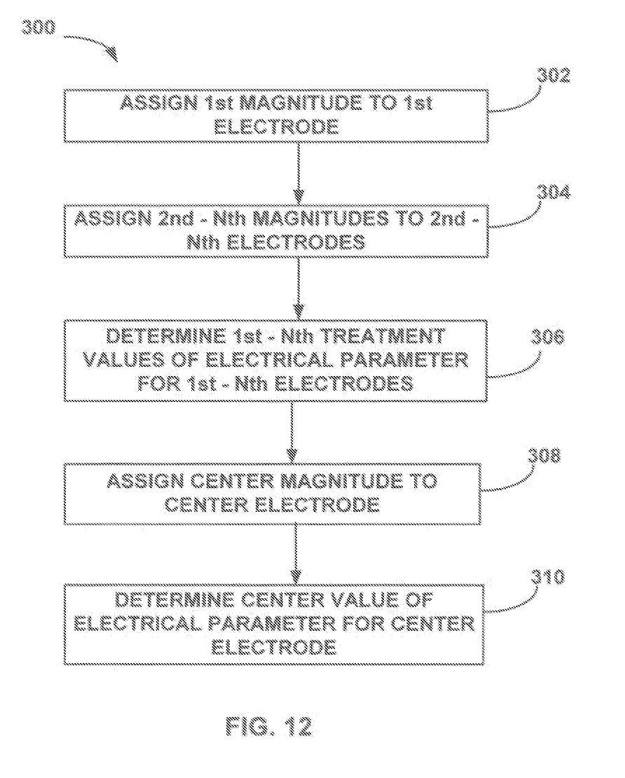

FIG. 13 is a flow chart schematically representing a series of steps involved in a method 400 for adjusting a value of a treatment parameter for an electrode unit, according to another embodiment of the invention. Method 400 may be performed during an electrosurgical procedure. As an example, such a procedure may result in lipolysis of targeted adipose tissue (e.g., body fat). Step 402 may involve monitoring at least a first electrical parameter of at least one electrode of the electrode unit. The electrode unit may comprise a plurality of concentric electrodes, and the at least one electrode may comprise at least one of the concentric electrodes. In an embodiment, the first electrical parameter may be current. In another embodiment, the first electrical parameter may be voltage.

Step 404 may involve moving the electrode unit with respect to a target tissue. As a non-limiting example, the target tissue may be adipose tissue, such as subcutaneous fat or fat deposits in combination with dermal fibrous tissue. In an embodiment, step 404 may involve moving the electrode unit in one or more planes substantially parallel to the patient's skin or a layer of subcutaneous fat (see, for example, FIGS. 9A-B). As an example, step 404 may involve moving the electrode unit over the surface of a patient's skin. During step 404, the electrode unit may be in contact with the patient's skin.

Step 406 may involve adjusting at least a second electrical parameter of the at least one electrode, in response to a change in the first electrical parameter. As an example, the second electrical parameter may be adjusted such that the second electrical parameter is at a suitable value, or within a suitable range, for effectively treating the target tissue. In an embodiment, the second electrical parameter may be current. In other embodiments, the second electrical parameter may be voltage. Step 408 may involve effectively treating the target tissue based on the suitably adjusted value, or range of values, of the second electrical parameter, e.g., such that an appropriate treatment temperature is attained within the target tissue.

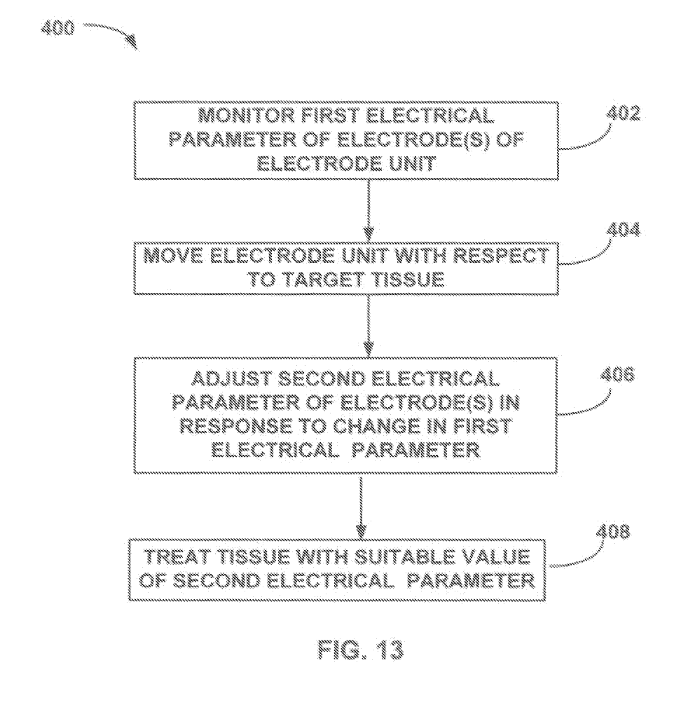

FIG. 14 is a flow chart schematically representing a series of steps involved in a method 500 for detecting thickness of a tissue, according to another embodiment of the invention. Step 502 may involve maintaining at least a first electrical parameter at a constant level for at least one electrode of an electrode unit. The electrode unit may comprise a plurality of concentric electrodes, and the at least one electrode may comprise at least one of the concentric electrodes. Method 500 may be performed before, during, or after an electrosurgical procedure, such as a procedure for decreasing the thickness of a layer of subcutaneous fat. For example, method 500 may be performed prior to treatment of subcutaneous fat to detect differences in thickness of the subcutaneous fat over different areas of a patient's body. As another example, method 500 may be performed during or after treatment to detect changes in thickness of subcutaneous fat, over different areas of a patient's body, resulting from treatment. In an embodiment, the first electrical parameter may be voltage. In another embodiment, the first electrical parameter may be current.

Step 504 may involve monitoring at least a second electrical parameter for the at least one electrode of the electrode unit. In an embodiment where the first electrical parameter may be current, the second electrical parameter may be voltage. In other embodiments where the first electrical parameter may be voltage, the second electrical parameter may be current. Step 504 may be performed concurrently with step 506.

Step 506 may involve moving the electrode unit with respect to a target tissue. The target tissue may comprise a layer of tissue, such as a layer of subcutaneous fat. Step 506 may involve moving the electrode unit in at least one direction substantially parallel to the layer of target tissue. Step 506 may be preformed substantially as described for step 404 of method 400 (FIG. 13, supra).

Step 508 may involve detecting a change in thickness of the target tissue based on at least one change in the second electrical parameter. As an example, step 508 may be performed while moving the electrode unit according to step 502, such that an operator (e.g., physician or other medical personnel) may spatially and/or temporally relate a change in the second electrical parameter, which is indicative of a change in thickness of the target tissue, to a particular region of the target tissue.

In an embodiment, step 506 of method 500 may be omitted. For example, steps 502, 504, and 508 may be performed to detect change in tissue thickness at a given location, e.g., to compare tissue thickness before and after a procedure, or to compare tissue thickness at different times, regardless of whether a procedure has been performed at that location.

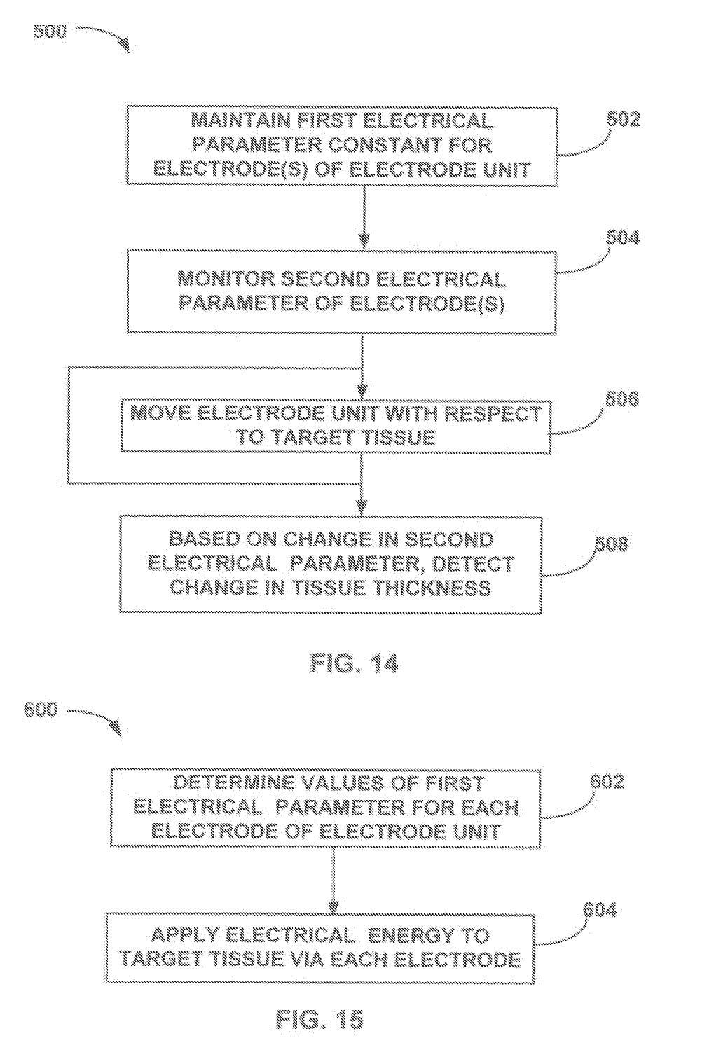

FIG. 15 is a flow chart schematically representing a series of steps involved in a method 600 for treating a target tissue, according to another embodiment of the invention. Step 602 may involve determining a treatment value of an electrical parameter for each of a plurality of concentric electrodes of an electrode unit. In an embodiment, each concentric electrode has a different pre-set value of the electrical parameter. As an example, the treatment value for each concentric electrode may be determined according to method 300 (FIG. 12, supra). The electrical parameter may be current, voltage, or power. Step 604 may involve applying electrical energy to the target tissue via each of the electrodes according to the treatment value determined in step 602.

Temperature Profiles of Selectively Heated Tissue

The applicant has found that electrode edge effects, which are typical of conventional electrode configurations, such as solid electrodes of the prior art, and which result in uneven heating of tissue and the production of hot spots, may be minimized if not eliminated by independently controlling energy to each of a plurality of concentric electrodes of an electrode unit configured as disclosed. Modeling software (COMSOL FEM (COMSOL, Inc., Burlington, Mass., USA)) was found to be useful in investigating the temperature profiles of electrode treated tissue sections comprising various thicknesses of skin, subcutaneous fat, and muscle tissues. A temperature profile for such a tissue section, for a particular electrode configuration and set of electrical parameters for each electrode of the electrode configuration, is presented in the Example that follows.

### EXAMPLE

### Temperature Profile for Tissue Sections Comprising Skin, Fat, and Muscle Layers

The model (COMSOL FEM) presented in this Example was for an electrode unit having a non-annular center electrode, in the form of a rod or pin, surrounded by six (6) concentric annular electrodes. The radius of each annular electrode (i.e., the total distance from the center electrode), and the set current for each annular electrode are presented in Table 1. The tissue according to this Example comprised an upper 2 mm skin (dermal) layer, SK; an intermediate 5 mm thick layer of subcutaneous fat, SF; and a lower 45 mm layer of muscle tissue, MU.

The results, in the form of a temperature profile for the tissue section, are shown in FIG. 16. The X axis shows the radius of tissue (in mm), and the Y axis shows the distance (tissue depth) from the skin surface (also in mm). The location of the electrode unit is indicated in FIG. 16 by the filled area labeled as LE. Note that in FIG. 16, the tissue and the electrode unit are symmetrical about the point marked “0” on the X axis (abscissa), i.e., only the right side of the tissue and right side of the electrode unit are shown. (FIG. 16 is not intended to be drawn to scale.)

TABLE 1
Set Current Values for Each of a Plurality of Electrodes of a Computer
Modeled Electrode Unit

| | Radial distance from | |
|---|---|---|
| | center electrode | |
| Electrode | (inches) | Set current (Amps) |
| Center | 0 (zero) | 0.029 |
| 1^{st} annular | 0.5 | 0.286 |
| 2^{nd} annular | 1.0 | 0.429 |
| 3^{rd} annular | 1.5 | 0.543 |
| 4^{th} annular | 2.0 | 0.571 |
| 5^{th} annular | 2.5 | 0.600 |
| 6^{th} annular | 3.0 | 0.629 |

From an examination of FIG. 16, the model clearly shows that the subcutaneous fat layer is selectively heated via the electrode unit to the highest temperature, while the non-target skin and muscle layers are at much lower temperatures. In particular, a zone of maximum heating in the subcutaneous fat layer showed a temperature of >75° C., while a temperature of <46° C. was recorded for much of the skin and almost the entire muscle layer. The time period to provide the temperature profile shown in FIG. 16 is 10 seconds. The maximum temperature of the subcutaneous fat (ca. 75° C.) is up to about 25° C. higher than that required to induce lipolysis (ca. 50-55° C.) of adipose tissue. Those skilled in the art will recognize that the invention, including the electrode unit configuration, electrical parameters, treatment time, and the like, may be modified or adjusted, as appropriate, to provide an overall reduction in the amount of heating of the tissue section as seen in FIG. 16, thus ensuring that non-targeted skin and muscle tissues are undamaged, while still easily attaining a temperature sufficient to induce lipolysis of the subcutaneous fat tissue.

When various other configurations of concentric electrodes were similarly modeled (using COMSOL FEM software), generally similar temperature profiles were obtained when suitable values of electrical parameters, e.g., current, were applied to each electrode (see, e.g., FIG. 12). Further, generally similar temperature profiles were also obtained for various tissue sections, i.e., for multi-layered tissues having different thicknesses of subcutaneous fat and muscle beneath a dermal layer or skin.

Although the various embodiments have been described primarily with respect to the treatment of adipose tissue (fat) as a target tissue, the present invention may also be applicable to the treatment of other target tissues which may be disposed adjacent to various non-target tissues in addition to skin and muscle.

It should be understood, of course, that the foregoing relates to exemplary embodiments of the invention, none of the examples presented herein are to be construed as limiting the present invention in any way, and that modifications may be made without departing from the spirit and scope of the invention as set forth in the following claims.

What is claimed is:

1. An electrosurgical system for treating a patient, comprising:
a power supply; and
an electrode unit configured for coupling to said power supply, wherein:
said electrode unit comprises a plurality of concentric electrodes,
said power supply is configured for supplying electrical energy to each of said plurality of concentric electrodes of said electrode unit, and
said system is configured for independently controlling a first electrical parameter of said electrical energy supplied to each of said plurality of concentric electrodes, and said system is further configured for providing a different value of said first electrical parameter to each said concentric electrode.

2. The system of claim 1, wherein:
said power supply includes a plurality of amplifiers, and
said system is configured for independently controlling supply of said electrical energy from each of said plurality of amplifiers to a corresponding one of said plurality of concentric electrodes.

3. The system of claim 2, wherein said plurality of concentric electrodes comprises a plurality of annular electrodes.

4. The system of claim 3, wherein said plurality of concentric electrodes further comprises an axially disposed center electrode, and wherein said center electrode is non-annular.

5. The system of claim 1, wherein:
said electrode unit includes a treatment face configured for contacting the patient's body,
said electrode unit is configured for providing a zone of maximum heating within a target tissue of the patient's body, and
the zone of maximum heating is located at a distance of at least about 3 mm from said treatment face.

6. The system of claim 1, wherein:
said electrode unit further comprises a treatment face adapted for contacting a patient's skin,
each of said plurality of concentric electrodes has a bare metal external surface, and
said treatment face comprises said bare metal external surface.

7. The system of claim 3, wherein said electrode unit comprises from about six (6) to about fifteen (15) of said annular electrodes.

8. The system of claim 3, wherein said electrode unit comprises from about five (5) to about twenty five (25) of said annular electrodes.

9. The system of claim 1, wherein said power supply is configured for independently supplying radiofrequency (RF) electrical energy to each of said plurality of concentric electrodes at a frequency in the range of from about 200 KHz to 3 MHz.

10. The system of claim 1, wherein said electrode unit comprises a direct-coupled electrode and at least one indirect-coupled electrode, said electrode unit further comprises at least one passive electrical element, and wherein each said indirect-coupled electrode is operably coupled to said direct-coupled electrode via one of said passive electrical elements such that said electrical energy is distributed from said direct-coupled electrode to said indirect-coupled electrodes in a defined manner.

11. The system of claim 10, wherein:
said passive electrical element comprises a capacitor, an inductor, or a resistor, and
said electrical energy is distributed from said direct-coupled electrode to said at least one indirect-coupled electrode according to a value of capacitance, inductance, or resistance of said at least one passive electrical element.

12. The system of claim 1, wherein:
said electrode unit further comprises an electrode perimeter,
each of said plurality of concentric electrodes comprises an electrically conductive metal, and
said electrode perimeter is a bare metal external surface.

13. A system for treating a patient, comprising:
an electrode unit including a plurality of concentric electrodes; and
a power supply including a plurality of amplifiers, wherein:
said electrode unit is configured for electrically coupling each of said plurality of concentric electrodes to a corresponding one of said plurality of amplifiers, and
said power supply is configured for independently controlling supply of electrical energy to each of said plurality of concentric electrodes from said corresponding one of said plurality of amplifiers.

14. The system of claim 13, wherein said plurality of concentric electrodes includes a non-annular center electrode disposed axially with respect to said electrode unit.

15. The system of claim 13, wherein said electrode unit is substantially disc-shaped.

16. A system for treating a patient, comprising:
a power supply; and
an electrode unit including a plurality of concentric electrodes, wherein:
said plurality of concentric electrodes include a direct-coupled electrode and a plurality of indirect-coupled electrodes,
said electrode unit is configured for direct electrical coupling of said direct-coupled electrode to said power supply,
said electrode unit is further configured for electrical coupling of said direct-coupled electrode to each of said indirect-coupled electrodes,
said power supply is configured for providing a supply of electrical energy to said electrode unit, and
said system is configured for independently controlling said supply of electrical energy from said at least one direct-coupled electrode to each of said indirect-coupled electrodes.

17. The system of claim 16, wherein said direct-coupled electrode comprises a non-annular center electrode or an annular electrode.

18. The system of claim 16, wherein said indirect-coupled electrodes comprise at least one annular electrode or a non-annular center electrode.

19. The system of claim 16, further comprising a plurality of passive electrical elements, wherein each of said indirect-coupled electrodes is in electrical communication with said direct-coupled electrode via a corresponding one of said passive electrical elements, wherein each of said passive electrical elements comprises a capacitor, an inductor, a resistor, or a combination thereof.

20. A system comprising:
a power supply; and
an electrode unit operably coupled to said power supply, said electrode unit including:
a plurality of concentric electrodes, and
a plurality of passive electrical elements, wherein each of said electrodes is in electrical communication with said power supply via a corresponding one of said passive electrical elements, such that said system is configured for providing a different value of a first electrical parameter of electrical energy to each said electrode.

21. The system of claim 20, wherein each of said plurality of passive electrical elements comprises at least one capacitor, at least one inductor, at least one resistor, or a combination thereof.

22. The system of claim 20, wherein each of said plurality of passive electrical elements has a different value of capacitance, inductance, or resistance.

23. The system of claim 20, wherein:
said electrode unit comprises from about six (6) to about fifteen (15) of said passive electrical elements, and
said plurality of concentric electrodes include from about six (6) to about fifteen (15) annular electrodes.

24. Apparatus comprising: an electrode unit including a plurality of concentric annular electrodes, and a non-annular center electrode arranged concentrically with respect to each of said plurality of annular electrodes.

25. The apparatus of claim 24, wherein said electrode unit includes from at least about 5 of said annular electrodes.

26. The apparatus of claim 24, wherein said electrode unit includes from about 6 to 15 of said annular electrodes.

27. The apparatus of claim 24, wherein:
said electrode unit is configured for contacting tissue of a patient, and
said electrode unit is further configured for avoiding capacitive coupling and inductive coupling of said electrode unit to said tissue.

28. The apparatus of claim 24, wherein said center electrode comprises a rod, a pin, or a post.

29. The apparatus of claim 24, wherein each of said plurality of annular electrodes lies in the same plane.

30. The apparatus of claim 24, wherein:
said electrode unit includes an electrode perimeter,
each of said plurality of concentric electrodes comprises an electrically conductive metal, and
said electrode perimeter is a bare metal external surface.

31. The apparatus of claim 24, further comprising a treatment face, wherein:
said treatment face is configured for contacting a patient's body,
said external surface of each of said plurality of concentric electrodes comprises a bare metal external surface, and
said treatment face comprises said bare metal external surface of each of said plurality of concentric electrodes.

32. An apparatus, comprising: an electrode unit including a plurality of concentric electrodes, wherein:
said plurality of concentric electrodes include a direct-coupled electrode and a plurality of indirect-coupled electrodes,
said electrode unit is configured for direct electrical coupling of said power supply to said direct-coupled electrode,
said electrode unit is further configured for electrical coupling of said direct-coupled electrode to each of said indirect-coupled electrodes, and
said system is configured for independently controlling supply of electrical energy from said at least one direct-coupled electrode to each of said indirect-coupled electrodes.

33. The apparatus of claim 32, wherein said direct-coupled electrode comprises a non-annular center electrode.

34. The apparatus of claim 32, wherein said indirect-coupled electrodes comprise at least one annular electrode.

35. The apparatus of claim 32, wherein said direct-coupled electrode comprises an annular electrode.

36. The apparatus of claim 32, wherein said indirect-coupled electrodes include a non-annular center electrode.

37. The apparatus of claim 32, further comprising a plurality of passive electrical elements, wherein each of said indirect-coupled electrodes is in electrical communication with said direct-coupled electrode via a corresponding one of said passive electrical elements.

38. The apparatus of claim 37, wherein:
each of said plurality of passive electrical elements comprises at least one capacitor, at least one inductor, at least one resistor, or a combination thereof.

39. The apparatus of claim 37, wherein each of said plurality of passive electrical elements has a different value of capacitance, inductance, or resistance.

40. The apparatus of claim 32, wherein said system is further configured for providing a different value of a first electrical parameter of said electrical energy to each said indirect-coupled electrode.

41. A handpiece, comprising:
an electrode unit adapted for treating tissue of a patient, wherein said electrode unit includes:
a plurality of concentric electrodes, and
a treatment face configured for contacting said patient, wherein:
each of said plurality of concentric electrodes comprises a bare metal external surface, and
said treatment face comprises said bare metal external surface.

42. The handpiece of claim 41, further comprising a housing, wherein said electrode unit is affixed to or integral with said housing.

43. The handpiece of claim 41, wherein said treatment face is rigid.

44. The handpiece of claim 41, wherein said treatment face is convex.

45. The handpiece of claim 41, wherein said treatment face is at least substantially planar.

46. The handpiece of claim 41, wherein said electrode unit comprises at least about five (5) of said concentric electrodes

47. The handpiece of claim 41, wherein said plurality of concentric electrodes comprises a plurality of annular electrodes.

48. The handpiece of claim 41, wherein said plurality of concentric electrodes comprises a non-annular center electrode.

49. The handpiece of claim 41, wherein said electrode unit further includes a dielectric spacer disposed between at least two of said concentric electrodes, and wherein said dielectric spacer comprises at least one spoke.

50. A method for treating a target tissue, comprising:
a) determining a treatment value of a first electrical parameter for each of a plurality of concentric electrodes of an electrode unit, wherein each said concentric electrode has a different value of said first electrical parameter; and
b) applying electrical energy to the target tissue via each said concentric electrode according to said treatment values determined in step a).

51. The method of claim 50, further comprising:
c) during step b), monitoring said first electrical parameter for at least one of said plurality of concentric electrodes; and
d) during step c), adjusting a second electrical parameter for said at least one concentric electrode in response to a change in said first electrical parameter.

52. The method of claim 51, further comprising:
e) moving said electrode unit with respect to the target tissue, wherein step e) is performed during at least one of steps b), c), and d).

53. The method of claim 50, further comprising:
f) maintaining said first electrical parameter at a constant level for at least one of said plurality of concentric electrodes;
g) during step f), moving said electrode unit with respect to the target tissue;
h) during step g), monitoring a second electrical parameter for said at least one concentric electrode; and
i) based on at least one change in said second electrical parameter, detecting a change in thickness of the target tissue.

54. The method of claim 50, wherein said first electrical parameter comprises current, voltage, or power.

55. The method of claim 50, wherein step b) comprises applying radiofrequency (RF) electrical energy to the target tissue at a frequency in the range of from about 200 KHz to 3 MHz.

56. A method for treating a patient, comprising:
a) disposing an electrode unit in relation to the patient's body, wherein:
said electrode unit comprises a plurality of concentric electrodes,
said electrode unit is electrically coupled to a power supply,
said power supply includes a plurality of amplifiers, and
each of said plurality of amplifiers is electrically coupled to a corresponding one of said plurality of concentric electrodes; and
b) while said electrode unit is disposed according to step a), selectively heating, via said plurality of concentric electrodes, a target tissue of the patient's body, wherein step b) comprises independently controlling supply of electrical energy, via said plurality of amplifiers, to each of said plurality of concentric electrodes.

57. The method of claim 56, wherein:
step a) comprises disposing said electrode unit on a non-target tissue, and
the target tissue is disposed distal to the non-target tissue and distal to said electrode unit.

58. The method of claim 56, wherein:
said electrode unit includes a treatment face configured for contacting the patient's body, and
a zone of maximum heating within the target tissue is located at a distance of at least about 3 mm from said treatment face.

59. The method of claim 57, wherein:
the non-target tissue comprises skin, and
the target tissue comprises subcutaneous fat.

60. The method of claim 56, wherein said electrode unit is configured for non-uniform heating of tissue in a Y dimension, wherein said Y dimension is substantially orthogonal to a plane substantially parallel to the target tissue, such that the target tissue is selectively heated relative to a non-target tissue, and said electrode unit is further configured for substantially uniform heating of the target tissue in an X dimension and a Z dimension, wherein said X and Z dimensions are in said plane substantially parallel to the target tissue.

61. The method of claim 56, wherein said electrode unit is monopolar.

62. A method for performing a procedure, comprising:
a) disposing an electrode unit at a treatment area of a patient's body, wherein said electrode unit comprises a plurality of concentric electrodes; and
b) via said electrode unit, applying electrical energy to a target tissue, wherein the target tissue is located beneath said treatment area, wherein step b) comprises independently controlling a first electrical parameter of said electrical energy supplied to each of said plurality of concentric electrodes, and wherein each said concentric electrode receives a different value of said first electrical parameter.

63. The method of claim 62, wherein:
said electrode unit is operably coupled to a power supply,
said power supply includes a plurality of amplifiers,
each of said plurality of concentric electrodes is independently coupled to a corresponding one of said plurality of amplifiers, and
step b) comprises independently controlling said first electrical parameter via said plurality of amplifiers.

64. The method of claim 62, wherein step b) comprises applying radiofrequency (RF) electrical energy to the target tissue at a frequency in the range of from about 300 KHz to 650 KHz.

65. The method of claim 62, wherein:
step a) comprises disposing said electrode unit on the patient's skin,
the target tissue comprises subcutaneous fat,
said electrical energy applied via said electrode unit is sufficient to cause lipolysis of at least a portion of adipocytes of the subcutaneous fat,
said electrode unit is configured for selectively heating the subcutaneous fat while said electrode unit is disposed on the patient's skin, and
said electrode unit is further configured for minimizing heating of the patient's skin during step b).

66. The method of claim 62, further comprising:
c) determining a treatment value of said first electrical parameter for each of said plurality of concentric electrodes, wherein each of said plurality of concentric electrodes has a different value of said first electrical parameter.

67. The method of claim 62, wherein:
said electrode unit comprises a treatment face,
step a) comprises contacting the patient's skin with said treatment face, and
said treatment face comprises a bare metal external surface of at least one of said plurality of concentric electrodes.

68. The method of claim 63, wherein during step b) at least a portion of the subcutaneous fat is heated to a temperature in the range of at least 50° C., and wherein during step b), the patient's skin is heated to a temperature of not more than 44° C.

69. A method for determining a treatment value of an electrical parameter for each of a plurality of electrodes of an electrode unit, the method comprising:
a) assigning a first magnitude, M₁, to a first electrode of said plurality of electrodes;
b) assigning a second through n^{th} magnitude, M₂-Mₙ, for each of a second through n^{th} electrode of said plurality of electrodes, wherein each of said second through n^{th} magnitudes is derived from said first magnitude; and
c) determining a first through n^{th} value, P₁-Pₙ, of said electrical parameter for a corresponding one of said first through n^{th} electrodes, wherein each of said first through n^{th} values, P₁-Pₙ, is a function of a corresponding one of said first through n^{th} magnitudes M₁-Mₙ.

70. The method of claim 69, wherein said electrical parameter is voltage, current, or power.

71. The method of claim 69, wherein:
said plurality of electrodes includes a plurality of annular electrodes,
said plurality of electrodes are configured concentrically with respect to each other,
said first electrode is radially innermost of said plurality of annular electrodes,
said second electrode is disposed radially outward from said first electrode, and
said n^{th} electrode is disposed radially outward from said second electrode.

72. The method of claim 69, wherein each of said second through nth magnitudes is derived with respect to each of said first through n^{th} magnitudes.

73. The method of claim 69, wherein said electrical parameter is current, and wherein values of said current, I₁-Iₙ for each of said first through n^{th} electrodes, respectively, are related to said magnitudes, M₁-Mₙ, by the relationship:
Iₓ=(Mₓ/Aₓ)*S,
wherein x denotes a particular one of said first through n^{th} electrodes, Iₓ is current for the particular one of said first through n^{th} electrodes, Mₓ is magnitude for the particular one of said first through n^{th} electrodes, the particular one of said first through n^{th} electrodes is an annular electrode, Aₓ is the area of a circle defined by the particular one of said first through n^{th} electrodes, and S is a scaling factor.

74. The method of claim 71, wherein:
step c) comprises determining a second value, P₂, of said electrical parameter for said second annular electrode,
said electrical parameter is voltage, and wherein P₁>P₂>Pₙ.

75. A method for adjusting a treatment parameter of an electrode unit, the method comprising:
a) monitoring at least a first electrical parameter of at least one electrode of said electrode unit; and
b) adjusting at least a second electrical parameter of said at least one electrode in response to a change in said first electrical parameter.

76. The method of claim 75, wherein:
said electrode unit comprises a plurality of concentric electrodes,
said at least one electrode comprises at least one of said plurality of concentric electrodes,
step a) comprises monitoring said first electrical parameter of each of said plurality of concentric electrodes, and
step b) comprises adjusting said second electrical parameter for each of said plurality of concentric electrodes.

77. The method of claim 75, wherein said first electrical parameter comprises voltage and said second electrical parameter comprises current.

78. The method of claim 75, wherein said first electrical parameter comprises current and said second electrical parameter comprises voltage.

79. The method of claim 75, further comprising:
c) during steps a) and b), disposing said electrode unit at a treatment area of a patient's body.

80. The method of claim 75, further comprising:
d) moving said electrode unit with respect to a target tissue, wherein:
the target tissue comprises a layer of tissue,
said moving step comprises moving said electrode unit in at least one direction substantially parallel to said layer, and
step b) is performed during step d).

81. A method for detecting tissue thickness, the method comprising:
a) maintaining at least a first electrical parameter at a constant level for at least one electrode of an electrode unit;
b) monitoring at least a second electrical parameter for said at least one electrode; and
c) based on at least one change in said second electrical parameter, detecting a change in thickness of a target tissue.

82. The method of claim 81, further comprising:
d) during steps a) and b), disposing said electrode unit with respect to the target tissue.

83. The method of claim 81, further comprising:
e) during step b), moving said electrode unit with respect to the target tissue.

84. The method of claim 83, wherein:
the target tissue comprises a layer of subcutaneous fat, and
step e) comprises moving said electrode unit in a direction at least substantially parallel to the layer of subcutaneous fat.

85. The method of claim 81, wherein said first electrical parameter comprises current and said second electrical parameter comprises voltage.

86. The method of claim 81, wherein said first electrical parameter comprises voltage and said second electrical parameter comprises current.

87. The method of claim 81, wherein:
said electrode unit comprises a plurality of annular electrodes,
each of said plurality of annular electrodes has a different value of said first electrical parameter, and
step a) comprises maintaining each of said plurality of annular electrodes at said different value of said first electrical parameter.
